SEQUENCE LISTING (156 sequences)

SEQ ID NO: 1 - DNA, 21 bp, synthetic construct
  misc_feature 1..21: note = Target sequence
  source 1..21: mol_type = other DNA
cccgagtcacagcaatccgga

SEQ ID NO: 2 - DNA, 21 bp, synthetic construct
  misc_feature 1..21: note = target sequence
  source 1..21: mol_type = other DNA
cccgagccgcagcagtcccga

SEQ ID NO: 3 - DNA, 64 bp, synthetic construct
  misc_feature 1..64: note = sequence encoding shRNA
  misc_feature 64: note = polyT
  source 1..64: mol_type = other DNA
agctttgtttgcccgagtcacagcaatccggattcaagagatccggattgctgtgactcgggcc

SEQ ID NO: 4 - RNA, 53 nt, synthetic construct
  source 1..53: mol_type = other RNA
  misc_feature 1..53: note = shRNA
gcccgagtcacagcaatccggattcaagagatccggattgctgtgactcgggc

SEQ ID NO: 5 - DNA, 18 bp, synthetic construct
  misc_feature 1..18: note = target sequence
  source 1..18: mol_type = other DNA
gatgagcctcaacgagca

SEQ ID NO: 6 - DNA, 58 bp, synthetic construct
  misc_feature 1..58: note = sequence encoding shRNA
  misc_feature 58: note = polyT
  source 1..58: mol_type = other DNA
agctttgtttggatgagcctcaacgagcattcaagagatgctcgttgaggctcatccc

SEQ ID NO: 7 - RNA, 47 nt, synthetic construct
  source 1..47: mol_type = other RNA
  misc_feature 1..47: note = shRNA
ggatgagcctcaacgagcattcaagagatgctcgttgaggctcatcc

SEQ ID NO: 8 - DNA, 28 bp, synthetic construct
  misc_feature 1..28: note = primer
  source 1..28: mol_type = other DNA
gcaggtaggagagtgtacaggatgcctt

SEQ ID NO: 9 - DNA, 23 bp, synthetic construct
  misc_feature 1..23: note = primer
  source 1..23: mol_type = other DNA
ctttccatccccactgccatttt

SEQ ID NO: 10 - DNA, 21 bp, synthetic construct
  misc_feature 1..21: note = primer
  source 1..21: mol_type = other DNA
atgcctactgccaaaattgac

SEQ ID NO: 11 - DNA, 22 bp, synthetic construct
  misc_feature 1..22: note = primer
  source 1..22: mol_type = other DNA
aaatactgagccatctctcctg

SEQ ID NO: 12 - DNA, 21 bp, synthetic construct
  misc_feature 1..21: note = primer
  source 1..21: mol_type = other DNA
aagccgtcatcgtgaagaaca

SEQ ID NO: 13 - DNA, 18 bp, synthetic construct
  misc_feature 1..18: note = primer
  source 1..18: mol_type = other DNA
gccaagctcaaagcttcc

SEQ ID NO: 14 - DNA, 23 bp, synthetic construct
  misc_feature 1..23: note = primer
  source 1..23: mol_type = other DNA
gcagctacctggaactcgtcacg

SEQ ID NO: 15 - DNA, 18 bp, synthetic construct
  misc_feature 1..18: note = primer
  source 1..18: mol_type = other DNA
gggccagtcagcactctc

SEQ ID NO: 16 - DNA, 20 bp, synthetic construct
  misc_feature 1..20: note = primer
  source 1..20: mol_type = other DNA
ctattcctgcgtcggtgtgt

SEQ ID NO: 17 - DNA, 20 bp, synthetic construct
  misc_feature 1..20: note = primer
  source 1..20: mol_type = other DNA
ccactcaaatcgaacacggc

SEQ ID NO: 18 - DNA, 23 bp, synthetic construct
  misc_feature 1..23: note = primer
  source 1..23: mol_type = other DNA
ctatgaacaggagaaggatgaag

SEQ ID NO: 19 - DNA, 20 bp, synthetic construct
  misc_feature 1..20: note = primer
  source 1..20: mol_type = other DNA
cgtcctggacaagaccaagt

SEQ ID NO: 20 - DNA, 20 bp, synthetic construct
  misc_feature 1..20: note = primer
  source 1..20: mol_type = other DNA
ccttgccctacagctgagtc

SEQ ID NO: 21 - DNA, 21 bp, synthetic construct
  misc_feature 1..21: note = primer
  source 1..21: mol_type = other DNA
cttgatcttggatcgcttggc

SEQ ID NO: 22 - DNA, 20 bp, synthetic construct
  misc_feature 1..20: note = primer
  source 1..20: mol_type = other DNA
cctggtgggtagtcatggtc

SEQ ID NO: 23 - DNA, 24 bp, synthetic construct
  misc_feature 1..24: note = primer
  source 1..24: mol_type = other DNA
gagggtactggtacttggtctcca

SEQ ID NO: 24 - DNA, 20 bp, synthetic construct
  misc_feature 1..20: note = primer
  source 1..20: mol_type = other DNA
agtggtgcaggcactaaatg

SEQ ID NO: 25 - DNA, 20 bp, synthetic construct
  misc_feature 1..20: note = primer
  source 1..20: mol_type = other DNA
attccaagtgcgtccgatga

SEQ ID NO: 26 - DNA, 20 bp, synthetic construct
  misc_feature 1..20: note = primer
  source 1..20: mol_type = other DNA
agattccgcctcctttctgc

SEQ ID NO: 27 - DNA, 18 bp, synthetic construct
  misc_feature 1..18: note = primer
  source 1..18: mol_type = other DNA
actcagaagccgaaggtt

SEQ ID NO: 28 - DNA, 20 bp, synthetic construct
  misc_feature 1..20: note = primer
  source 1..20: mol_type = other DNA
attgctgtcccgaatgtctc

SEQ ID NO: 29 - DNA, 20 bp, synthetic construct
  misc_feature 1..20: note = primer
  source 1..20: mol_type = other DNA
cacactctcccccacattct

SEQ ID NO: 30 - DNA, 20 bp, synthetic construct
  misc_feature 1..20: note = primer
  source 1..20: mol_type = other DNA
tacggtaaactgcccacttg

SEQ ID NO: 31 - DNA, 20 bp, synthetic construct
  misc_feature 1..20: note = primer
  source 1..20: mol_type = other DNA
aggaaagtcccataaggtca

SEQ ID NO: 32 - DNA, 70 bp, synthetic construct
  misc_feature 1..70: note = Scr1_Fw
  source 1..70: mol_type = other DNA
agctttgtttacgacgtcacggcagcgatcacttcaagagagtgatcgctgccgtgacgtcgtcttttta

SEQ ID NO: 33 - DNA, 70 bp, synthetic construct
  misc_feature 1..70: note = Scr1_Rv
  source 1..70: mol_type = other DNA
gatctaaaaagacgacgtcacggcagcgatcactctcttgaagtgatcgctgccgtgacgtcgtaaacaa

SEQ ID NO: 34 - DNA, 70 bp, synthetic construct
  misc_feature 1..70: note = Sh22_Fw
  source 1..70: mol_type = other DNA
agctttgtttgcccgagtcacagcaatccggattcaagagatccggattgctgtgactcgggccttttta

SEQ ID NO: 35 - DNA, 70 bp, synthetic construct
  misc_feature 1..70: note = Sh22_Rv
  source 1..70: mol_type = other DNA
gatctaaaaaggcccgagtcacagcaatccggatctcttgaatccggattgctgtgactcgggcaaacaa

SEQ ID NO: 36 - DNA, 64 bp, synthetic construct
  misc_feature 1..64: note = Scr19_Fw
  source 1..64: mol_type = other DNA
agctttgttttaggcgcgtcacgaagaacttcaagagagttcttcgtgacgcgcctacttttta

SEQ ID NO: 37 - DNA, 64 bp, synthetic construct
  misc_feature 1..64: note = Scr19_Rv
  source 1..64: mol_type = other DNA
gatctaaaaagtaggcgcgtcacgaagaactctcttgaagttcttcgtgacgcgcctaaaacaa

SEQ ID NO: 38 - DNA, 64 bp, synthetic construct
  misc_feature 1..64: note = Sh190_Fw
  source 1..64: mol_type = other DNA
agctttgtttggatgagcctcaacgagcattcaagagatgctcgttgaggctcatcccttttta

SEQ ID NO: 39 - DNA, 64 bp, synthetic construct
  misc_feature 1..64: note = Sh190_Rv
  source 1..64: mol_type = other DNA
gatctaaaaagggatgagcctcaacgagcatctcttgaatgctcgttgaggctcatccaaacaa

SEQ ID NO: 40 - DNA, 70 bp, synthetic construct
  misc_feature 1..70: note = Scr22_Fw
  source 1..70: mol_type = other DNA
agctttgttttcttatgcggtattctctcttcttcaagagagaagagagaataccgcataagacttttta

SEQ ID NO: 41 - DNA, 70 bp, synthetic construct
  misc_feature 1..70: note = Scr22_Rv
  source 1..70: mol_type = other DNA
gatctaaaaagtcttatgcggtattctctcttctctcttgaagaagagagaataccgcataagaaaacaa

SEQ ID NO: 42 - DNA, 70 bp, synthetic construct
  misc_feature 1..70: note = Sh760_Fw
  source 1..70: mol_type = other DNA
agctttgtttatcgtctttgctgttcacttctttcaagagaagaagtgaacagcaaagacgatcttttta

SEQ ID NO: 43 - DNA, 70 bp, synthetic construct
  misc_feature 1..70: note = Sh760_Rv
  source 1..70: mol_type = other DNA
gatctaaaaagatcgtctttgctgttcacttcttctcttgaaagaagtgaacagcaaagacgataaacaa

SEQ ID NO: 44 - DNA, 70 bp, synthetic construct
  misc_feature 1..70: note = Scr4_Fw
  source 1..70: mol_type = other DNA
agctttgtttgaccacacagtcgcgctataccttcaagagaggtatagcgcgactgtgtggtccttttta

SEQ ID NO: 45 - DNA, 70 bp, synthetic construct
  misc_feature 1..70: note = Scr4_Rv
  source 1..70: mol_type = other DNA
gatctaaaaaggaccacacagtcgcgctatacctctcttgaaggtatagcgcgactgtgtggtcaaacaa

SEQ ID NO: 46 - DNA, 70 bp, synthetic construct
  misc_feature 1..70: note = Sh894_Fw
  source 1..70: mol_type = other DNA
agctttgtttaccgggcacccactatgcctaattcaagagattaggcatagtgggtgcccggtcttttta

SEQ ID NO: 47 - DNA, 70 bp, synthetic construct
  misc_feature 1..70: note = Sh894_Rv
  source 1..70: mol_type = other DNA
gatctaaaaagaccgggcacccactatgcctaatctcttgaattaggcatagtgggtgcccggtaaacaa

SEQ ID NO: 48 - DNA, 19 bp, synthetic construct
  misc_feature 1..19: note = target sequence
  source 1..19: mol_type = other DNA
ggactggatcggccagagt

SEQ ID NO: 49 - DNA, 19 bp, synthetic construct
  misc_feature 1..19: note = target sequence
  source 1..19: mol_type = other DNA
ccgaggtgatgagcctcaa

SEQ ID NO: 50 - DNA, 19 bp, synthetic construct
  misc_feature 1..19: note = target sequence
  source 1..19: mol_type = other DNA
cctgcatcctgcccaacat

SEQ ID NO: 51 - DNA, 19 bp, synthetic construct
  misc_feature 1..19: note = target sequence
  source 1..19: mol_type = other DNA
gcatcctgcccaacatcga

SEQ ID NO: 52 - DNA, 19 bp, synthetic construct
  misc_feature 1..19: note = target sequence
  source 1..19: mol_type = other DNA
aggtgatgagcctcaacga

SEQ ID NO: 53 - DNA, 19 bp, synthetic construct
  misc_feature 1..19: note = target sequence
  source 1..19: mol_type = other DNA
tgtcctggcgcaagctcta

SEQ ID NO: 54 - DNA, 19 bp, synthetic construct
  misc_feature 1..19: note = target sequence
  source 1..19: mol_type = other DNA
cgccggagccgccgccgca

SEQ ID NO: 55 - DNA, 19 bp, synthetic construct
  misc_feature 1..19: note = target sequence
  source 1..19: mol_type = other DNA
gccgccgccgcagcgggga

SEQ ID NO: 56 - DNA, 19 bp, synthetic construct
  misc_feature 1..19: note = target sequence
  source 1..19: mol_type = other DNA
gccgcagcggggacgggga

SEQ ID NO: 57 - DNA, 19 bp, synthetic construct
  misc_feature 1..19: note = target sequence
  source 1..19: mol_type = other DNA
cgctgtcctggcgcaagct

SEQ ID NO: 58 - DNA, 19 bp, synthetic construct
  misc_feature 1..19: note = target sequence
  source 1..19: mol_type = other DNA
cctggcgcaagctctactt

SEQ ID NO: 59 - DNA, 19 bp, synthetic construct
  misc_feature 1..19: note = target sequence
  source 1..19: mol_type = other DNA
gcgcaagctctacttgagc

SEQ ID NO: 60 - DNA, 19 bp, synthetic construct
  misc_feature 1..19: note = target sequence
  source 1..19: mol_type = other DNA
gcccccgagccgcagcagt

SEQ ID NO: 61 - DNA, 19 bp, synthetic construct
  misc_feature 1..19: note = target sequence
  source 1..19: mol_type = other DNA
ccccgagccgcagcagtcc

SEQ ID NO: 62 - DNA, 19 bp, synthetic construct
  misc_feature 1..19: note = target sequence
  source 1..19: mol_type = other DNA
gccgcagcagtcccgagct

SEQ ID NO: 63 - DNA, 19 bp, synthetic construct
  misc_feature 1..19: note = target sequence
  source 1..19: mol_type = other DNA
ccgcagcagtcccgagctt

SEQ ID NO: 64 - DNA, 19 bp, synthetic construct
  misc_feature 1..19: note = target sequence
  source 1..19: mol_type = other DNA
cgcagcagtcccgagcttc

SEQ ID NO: 65 - DNA, 19 bp, synthetic construct
  misc_feature 1..19: note = target sequence
  source 1..19: mol_type = other DNA
cccccaagcggcggcagca

SEQ ID NO: 66 - DNA, 19 bp, synthetic construct
  misc_feature 1..19: note = target sequence
  source 1..19: mol_type = other DNA
ccccaagcggcggcagcac

SEQ ID NO: 67 - DNA, 19 bp, synthetic construct
  misc_feature 1..19: note = target sequence
  source 1..19: mol_type = other DNA
ggcagcaccaccagcggca

SEQ ID NO: 68 - DNA, 19 bp, synthetic construct
  misc_feature 1..19: note = target sequence
  source 1..19: mol_type = other DNA
gcccccgggggccccgcca

SEQ ID NO: 69 - DNA, 19 bp, synthetic construct
  misc_feature 1..19: note = target sequence
  source 1..19: mol_type = other DNA
gccccgccaccgccgccgt

SEQ ID NO: 70 - DNA, 19 bp, synthetic construct
  misc_feature 1..19: note = target sequence
  source 1..19: mol_type = other DNA
ccgccgccgtccgccgtca

SEQ ID NO: 71 - DNA, 19 bp, synthetic construct
  misc_feature 1..19: note = target sequence
  source 1..19: mol_type = other DNA
ccgccgtccgccgtcacct

SEQ ID NO: 72 - DNA, 19 bp, synthetic construct
  misc_feature 1..19: note = target sequence
  source 1..19: mol_type = other DNA
cgccgtccgccgtcaccta

SEQ ID NO: 73 - DNA, 19 bp, synthetic construct
  misc_feature 1..19: note = target sequence
  source 1..19: mol_type = other DNA
gccgtccgccgtcacctac

SEQ ID NO: 74 - DNA, 19 bp, synthetic construct
  misc_feature 1..19: note = target sequence
  source 1..19: mol_type = other DNA
ccgccgtcacctacccgga

SEQ ID NO: 75 - DNA, 19 bp, synthetic construct
  misc_feature 1..19: note = target sequence
  source 1..19: mol_type = other DNA
gccgtcacctacccggact

SEQ ID NO: 76 - DNA, 19 bp, synthetic construct
  misc_feature 1..19: note = target sequence
  source 1..19: mol_type = other DNA
cacctacccggactggatc

SEQ ID NO: 77 - DNA, 19 bp, synthetic construct
  misc_feature 1..19: note = target sequence
  source 1..19: mol_type = other DNA
ccggactggatcggccaga

SEQ ID NO: 78 - DNA, 19 bp, synthetic construct
  misc_feature 1..19: note = target sequence
  source 1..19: mol_type = other DNA
gactggatcggccagagtt

SEQ ID NO: 79 - DNA, 19 bp, synthetic construct
  misc_feature 1..19: note = target sequence
  source 1..19: mol_type = other DNA
actggatcggccagagtta

SEQ ID NO: 80 - DNA, 19 bp, synthetic construct
  misc_feature 1..19: note = target sequence
  source 1..19: mol_type = other DNA
gccagagttactccgaggt

SEQ ID NO: 81 - DNA, 19 bp, synthetic construct
  misc_feature 1..19: note = target sequence
  source 1..19: mol_type = other DNA
cagagttactccgaggtga

SEQ ID NO: 82 - DNA, 19 bp, synthetic construct
  misc_feature 1..19: note = target sequence
  source 1..19: mol_type = other DNA
gcctcaacgagcactccat

SEQ ID NO: 83 - DNA, 19 bp, synthetic construct
  misc_feature 1..19: note = target sequence
  source 1..19: mol_type = other DNA
tcaacgagcactccatgca

SEQ ID NO: 84 - DNA, 19 bp, synthetic construct
  misc_feature 1..19: note = target sequence
  source 1..19: mol_type = other DNA
cactccatgcaggcgctgt

SEQ ID NO: 85 - DNA, 19 bp, synthetic construct
  misc_feature 1..19: note = target sequence
  source 1..19: mol_type = other DNA
cttgagccgcgccaagctt

SEQ ID NO: 86 - DNA, 19 bp, synthetic construct
  misc_feature 1..19: note = target sequence
  source 1..19: mol_type = other DNA
tgagccgcgccaagcttaa

SEQ ID NO: 87 - DNA, 19 bp, synthetic construct
  misc_feature 1..19: note = target sequence
  source 1..19: mol_type = other DNA
gagccgcgccaagcttaaa

SEQ ID NO: 88 - DNA, 19 bp, synthetic construct
  misc_feature 1..19: note = target sequence
  source 1..19: mol_type = other DNA
cgcgccaagcttaaagcct

SEQ ID NO: 89 - DNA, 19 bp, synthetic construct
  misc_feature 1..19: note = target sequence
  source 1..19: mol_type = other DNA
gcgccaagcttaaagcctc

SEQ ID NO: 90 - DNA, 19 bp, synthetic construct
  misc_feature 1..19: note = target sequence
  source 1..19: mol_type = other DNA
cgccaagcttaaagcctcc

SEQ ID NO: 91 - DNA, 19 bp, synthetic construct
  misc_feature 1..19: note = target sequence
  source 1..19: mol_type = other DNA
gccaagcttaaagcctcca

SEQ ID NO: 92 - DNA, 19 bp, synthetic construct
  misc_feature 1..19: note = target sequence
  source 1..19: mol_type = other DNA
ccagccggacctcggctct

SEQ ID NO: 93 - DNA, 19 bp, synthetic construct
  misc_feature 1..19: note = target sequence
  source 1..19: mol_type = other DNA
gccggacctcggctctgct

SEQ ID NO: 94 - DNA, 19 bp, synthetic construct
  misc_feature 1..19: note = target sequence
  source 1..19: mol_type = other DNA
cggacctcggctctgctct

SEQ ID NO: 95 - DNA, 19 bp, synthetic construct
  misc_feature 1..19: note = target sequence
  source 1..19: mol_type = other DNA
cggctctgctctccggctt

SEQ ID NO: 96 - DNA, 19 bp, synthetic construct
  misc_feature 1..19: note = target sequence
  source 1..19: mol_type = other DNA
ggctctgctctccggcttc

SEQ ID NO: 97 - DNA, 19 bp, synthetic construct
  misc_feature 1..19: note = target sequence
  source 1..19: mol_type = other DNA
cggcttcgccatggtggca

SEQ ID NO: 98 - DNA, 19 bp, synthetic construct
  misc_feature 1..19: note = target sequence
  source 1..19: mol_type = other DNA
ggcttcgccatggtggcaa

SEQ ID NO: 99 - DNA, 19 bp, synthetic construct
  misc_feature 1..19: note = target sequence
  source 1..19: mol_type = other DNA
gcttcgccatggtggcaat

SEQ ID NO: 100 - DNA, 19 bp, synthetic construct
  misc_feature 1..19: note = target sequence
  source 1..19: mol_type = other DNA
ctggacgctgaccacgact

SEQ ID NO: 101 - DNA, 19 bp, synthetic construct
  misc_feature 1..19: note = target sequence
  source 1..19: mol_type = other DNA
cgctgaccacgactaccca

SEQ ID NO: 102 - DNA, 19 bp, synthetic construct
  misc_feature 1..19: note = target sequence
  source 1..19: mol_type = other DNA
cccaccggggctgctcatc

SEQ ID NO: 103 - DNA, 19 bp, synthetic construct
  misc_feature 1..19: note = target sequence
  source 1..19: mol_type = other DNA
ccggggctgctcatcgcct

SEQ ID NO: 104 - DNA, 19 bp, synthetic construct
  misc_feature 1..19: note = target sequence
  source 1..19: mol_type = other DNA
cggggctgctcatcgcctt

SEQ ID NO: 105 - DNA, 19 bp, synthetic construct
  misc_feature 1..19: note = target sequence
  source 1..19: mol_type = other DNA
ggggctgctcatcgccttc

SEQ ID NO: 106 - DNA, 19 bp, synthetic construct
  misc_feature 1..19: note = target sequence
  source 1..19: mol_type = other DNA
gggctgctcatcgccttca

SEQ ID NO: 107 - DNA, 19 bp, synthetic construct
  misc_feature 1..19: note = target sequence
  source 1..19: mol_type = other DNA
gctgctcatcgccttcagt

SEQ ID NO: 108 - DNA, 19 bp, synthetic construct
  misc_feature 1..19: note = target sequence
  source 1..19: mol_type = other DNA
gccttcagtgcctgcacca

SEQ ID NO: 109 - DNA, 19 bp, synthetic construct
  misc_feature 1..19: note = target sequence
  source 1..19: mol_type = other DNA
cctgcaccacagtgctggt

SEQ ID NO: 110 - DNA, 19 bp, synthetic construct
  misc_feature 1..19: note = target sequence
  source 1..19: mol_type = other DNA
ccacagtgctggtggctgt

SEQ ID NO: 111 - DNA, 19 bp, synthetic construct
  misc_feature 1..19: note = target sequence
  source 1..19: mol_type = other DNA
ctggtggctgtgcacctgt

SEQ ID NO: 112 - DNA, 19 bp, synthetic construct
  misc_feature 1..19: note = target sequence
  source 1..19: mol_type = other DNA
ggtggctgtgcacctgttt

SEQ ID NO: 113 - DNA, 19 bp, synthetic construct
  misc_feature 1..19: note = target sequence
  source 1..19: mol_type = other DNA
gtgcacctgtttgcgctca

SEQ ID NO: 114 - DNA, 19 bp, synthetic construct
  misc_feature 1..19: note = target sequence
  source 1..19: mol_type = other DNA
cacctgtttgcgctcatga

SEQ ID NO: 115 - DNA, 19 bp, synthetic construct
  misc_feature 1..19: note = target sequence
  source 1..19: mol_type = other DNA
ctgtttgcgctcatgatca

SEQ ID NO: 116 - DNA, 19 bp, synthetic construct
  misc_feature 1..19: note = target sequence
  source 1..19: mol_type = other DNA
gcgctcatgatcagcacct

SEQ ID NO: 117 - DNA, 19 bp, synthetic construct
  misc_feature 1..19: note = target sequence
  source 1..19: mol_type = other DNA
gcacctgcatcctgcccaa

SEQ ID NO: 118 - DNA, 19 bp, synthetic construct
  misc_feature 1..19: note = target sequence
  source 1..19: mol_type = other DNA
cccaacatcgaggcggtga

SEQ ID NO: 119 - DNA, 19 bp, synthetic construct
  misc_feature 1..19: note = target sequence
  source 1..19: mol_type = other DNA
gcggtgagcaacgtgcaca

SEQ ID NO: 120 - DNA, 19 bp, synthetic construct
  misc_feature 1..19: note = target sequence
  source 1..19: mol_type = other DNA
cggtgagcaacgtgcacaa

SEQ ID NO: 121 - DNA, 19 bp, synthetic construct
  misc_feature 1..19: note = target sequence
  source 1..19: mol_type = other DNA
ggtgagcaacgtgcacaat

SEQ ID NO: 122 - DNA, 19 bp, synthetic construct
  misc_feature 1..19: note = target sequence
  source 1..19: mol_type = other DNA
tgagcaacgtgcacaatct

SEQ ID NO: 123 - DNA, 19 bp, synthetic construct
  misc_feature 1..19: note = target sequence
  source 1..19: mol_type = other DNA
gcaacgtgcacaatctcaa

SEQ ID NO: 124 - DNA, 19 bp, synthetic construct
  misc_feature 1..19: note = target sequence
  source 1..19: mol_type = other DNA
cacaatctcaactcggtca

SEQ ID NO: 125 - DNA, 19 bp, synthetic construct
  misc_feature 1..19: note = target sequence
  source 1..19: mol_type = other DNA
acaatctcaactcggtcaa

SEQ ID NO: 126 - DNA, 19 bp, synthetic construct
  misc_feature 1..19: note = target sequence
  source 1..19: mol_type = other DNA
ctcaactcggtcaaggagt

SEQ ID NO: 127 - DNA, 19 bp, synthetic construct
  misc_feature 1..19: note = target sequence
  source 1..19: mol_type = other DNA
cggtcaaggagtcccccca

SEQ ID NO: 128 - DNA, 19 bp, synthetic construct
  misc_feature 1..19: note = target sequence
  source 1..19: mol_type = other DNA
ggtcaaggagtccccccat

SEQ ID NO: 129 - DNA, 19 bp, synthetic construct
  misc_feature 1..19: note = target sequence
  source 1..19: mol_type = other DNA
ttgagattgtgcacgttgc

SEQ ID NO: 130 - DNA, 19 bp, synthetic construct
  misc_feature 1..19: note = target sequence
  source 1..19: mol_type = other DNA
attgtgcacgttgctcacc

SEQ ID NO: 131 - DNA, 19 bp, synthetic construct
  misc_feature 1..19: note = target sequence
  source 1..19: mol_type = other DNA
atcacctcggagtaactct

SEQ ID NO: 132 - DNA, 19 bp, synthetic construct
  misc_feature 1..19: note = target sequence
  source 1..19: mol_type = other DNA
atggagtgctcgttgaggc

SEQ ID NO: 133 - DNA, 19 bp, synthetic construct
  misc_feature 1..19: note = target sequence
  source 1..19: mol_type = other DNA
atcatgagcgcaaacaggt

SEQ ID NO: 134 - DNA, 19 bp, synthetic construct
  misc_feature 1..19: note = target sequence
  source 1..19: mol_type = other DNA
atgttgggcaggatgcagg

SEQ ID NO: 135 - DNA, 19 bp, synthetic construct
  misc_feature 1..19: note = target sequence
  source 1..19: mol_type = other DNA
aagtagagcttgcgccagg

SEQ ID NO: 136 - DNA, 19 bp, synthetic construct
  misc_feature 1..19: note = target sequence
  source 1..19: mol_type = other DNA
ctcatcacctcggagtaac

SEQ ID NO: 137 - DNA, 19 bp, synthetic construct
  misc_feature 1..19: note = target sequence
  source 1..19: mol_type = other DNA
agattgtgcacgttgctca

SEQ ID NO: 138 - DNA, 19 bp, synthetic construct
  misc_feature 1..19: note = target sequence
  source 1..19: mol_type = other DNA
agtaactctggccgatcca

SEQ ID NO: 139 - DNA, 19 bp, synthetic construct
  misc_feature 1..19: note = target sequence
  source 1..19: mol_type = other DNA
atgcgctcatggggggact

SEQ ID NO: 140 - DNA, 19 bp, synthetic construct
  misc_feature 1..19: note = target sequence
  source 1..19: mol_type = other DNA
gcaacgtgcacaatctcaa

SEQ ID NO: 141 - DNA, 19 bp, synthetic construct
  misc_feature 1..19: note = target sequence
  source 1..19: mol_type = other DNA
cggtgagcaacgtgcacaa

SEQ ID NO: 142 - DNA, 19 bp, synthetic construct
  misc_feature 1..19: note = target sequence
  source 1..19: mol_type = other DNA
gcaacgtgcacaatctcaa

SEQ ID NO: 143 - DNA, 19 bp, synthetic construct
  misc_feature 1..19: note = target sequence
  source 1..19: mol_type = other DNA
actggatcggccagagtta

SEQ ID NO: 144 - DNA, 19 bp, synthetic construct
  misc_feature 1..19: note = target sequence
  source 1..19: mol_type = other DNA
agagttactccgaggtgat

SEQ ID NO: 145 - DNA, 19 bp, synthetic construct
  misc_feature 1..19: note = target sequence
  source 1..19: mol_type = other DNA
gcctcaacgagcactccat

SEQ ID NO: 146 - DNA, 19 bp, synthetic construct
  misc_feature 1..19: note = target sequence
  source 1..19: mol_type = other DNA
ggcgcaagctctacttgag

SEQ ID NO: 147 - DNA, 19 bp, synthetic construct
  misc_feature 1..19: note = target sequence
  source 1..19: mol_type = other DNA
ggcttcgccatggtggcaa

SEQ ID NO: 148 - DNA, 19 bp, synthetic construct
  misc_feature 1..19: note = target sequence
  source 1..19: mol_type = other DNA
gcttcgccatggtggcaat

SEQ ID NO: 149 - DNA, 19 bp, synthetic construct
  misc_feature 1..19: note = target sequence
  source 1..19: mol_type = other DNA
tggacgctgaccacgacta

SEQ ID NO: 150 - DNA, 19 bp, synthetic construct
  misc_feature 1..19: note = target sequence
  source 1..19: mol_type = other DNA
cggggctgctcatcgcctt

SEQ ID NO: 151 - DNA, 19 bp, synthetic construct
  misc_feature 1..19: note = target sequence
  source 1..19: mol_type = other DNA
gcacctgcatcctgcccaa

SEQ ID NO: 152 - DNA, 19 bp, synthetic construct
  misc_feature 1..19: note = target sequence
  source 1..19: mol_type = other DNA
acatcgaggcggtgagcaa

SEQ ID NO: 153 - DNA, 19 bp, synthetic construct
  misc_feature 1..19: note = target sequence
  source 1..19: mol_type = other DNA
cggtgagcaacgtgcacaa

SEQ ID NO: 154 - DNA, 19 bp, synthetic construct
  misc_feature 1..19: note = target sequence
  source 1..19: mol_type = other DNA
ggtgagcaacgtgcacaat

SEQ ID NO: 155 - DNA, 19 bp, synthetic construct
  misc_feature 1..19: note = target sequence
  source 1..19: mol_type = other DNA
gcaacgtgcacaatctcaa

SEQ ID NO: 156 - DNA, 19 bp, synthetic construct
  misc_feature 1..19: note = target sequence
  source 1..19: mol_type = other DNA
acaatctcaactcggtcaa

(19) United States
(12) Patent Application Publication
(10) Pub. No.: US 2025/0163430 A1
(43) Pub. Date: May 22, 2025

(54) METHOD FOR TREATING TUBULAR AGGREGATE MYOPATHY AND STORMORKEN SYNDROME

(71) Applicants: UNIVERSITE DE STRASBOURG, STRASBOURG (FR); INSTITUT NATIONAL DE LA SANTÉ ET DE LA RECHERCHE MÉDICALE (INSERM), PARIS (FR); CENTRE NATIONAL AL DE LA RECHERCHE SCIENTIFIQUE, PARIS (FR); ASSOCIATION FRANCAISE CONTRE LES MYOPATHIES (AFM), Paris cedex 13 (FR)

(72) Inventors: JOCELYN LAPORTE, STRASBOURG (FR); JOHANN BOHM, STRASBOURG (FR); ROBERTO SILVA ROJAS, MADRID (ES)

(21) Appl. No.: 18/703,388

(22) PCT Filed: Oct. 21, 2022
(86) PCT No.: PCT/EP2022/079348

§ 371 Date: Apr. 22, 2024

(30) Foreign Application Priority Data
Oct. 22, 2021 (EP) 21306473.6

(51) Int. Cl. C12N 15/113; A61P 21/00; C12N 15/86

(52) U.S. Cl. CPC C12N 15/1138; A61P 21/00; C12N 15/86; C12N 2310/14; C12N 2310/531; C12N 2750/14143

(57) ABSTRACT

The present invention relates to the use of shRNA inhibiting ORAI1 expression for the treatment of a disease selected from the group consisting of tubular aggregate myopathy (TAM) and Stormorken syndrome (STRMK).

### FIELD OF THE INVENTION

The present invention relates to the field of medicine, in particular to the treatment of a disease selected from the group consisting of tubular aggregate myopathy (TAM) and Stormorken syndrome (STRMK).

### BACKGROUND OF THE INVENTION

ORAI1, ORAI2, and ORAI3 are broadly expressed and highly selective calcium (Ca²⁺) channels residing at the plasma membrane. Ca²⁺ is a universal second messenger and initiates a wide variety of conserved signaling cascades. It is primarily stored in the endoplasmic/sarcoplasmic reticulum (ER/SR), and the transient increase of cytosolic Ca²⁺ levels modulates transcription and mediates a multitude of biological processes including cell proliferation and motility, exocytosis, nerve conduction, hormone release, coagulation, and muscle contraction. Hence, the precise regulation of Ca²⁺ entry, Ca²⁺ storage, and Ca²⁺ release forms the basis for normal physiology in all cell types. One of the major mechanisms controlling Ca²⁺ homeostasis is store-operated Ca²⁺ entry (SOCE), which essentially relies on the concerted activity of the Ca²⁺ channel ORAI1 and the reticular Ca²⁺ sensor STIM1. Ca²⁺ store depletion from the ER/SR induces a conformational change of STIM1, resulting in protein oligomerization and the interaction with ORAI1 to trigger extracellular Ca²⁺ entry, ensure Ca²⁺ store refill, and maintain high Ca²⁺ gradients enabling oscillatory Ca²⁺ signaling.

Pathologic alterations of SOCE impeding or increasing Ca²⁺ influx profoundly compromise proper Ca²⁺ signaling and impact on various molecular, physiological, and biochemical functions in tissues and organs, leading to multi-systemic mirror diseases. Recessive STIM1 and ORAI1 loss-of-function (LoF) mutations inhibit SOCE and Ca²⁺ store refill, and cause severe combined immunodeficiency (SCID), characterized by recurrent and chronic infections, autoimmunity, muscular hypotonia, mydriasis, and amelogenesis imperfecta. By contrast, dominant STIM1 and ORAI1 gain-of-function (GoF) mutations inducing SOCE overactivity and excessive Ca²⁺ entry give rise to tubular aggregate myopathy (TAM) and Stormorken syndrome (STRMK), two clinically overlapping disorders associating childhood-onset muscle weakness with miosis, ichthyosis, short stature, hyposplenism, thrombocytopenia, and dyslexia (Bohm et al, 2013, Am J Hum Genet 92, 271-278; Bohm et al, 2018, Cell Calcium 76, 1-9; Endo et al, 2015, Hum Mol Genet 24, 637-648; Misceo et al, 2014, Hum Mutat 35, 556-564; Morin et al, 2020, Hum Mutat 41, 17-37; Morin et al, 2014, Hum Mutat 35, 1221-1232; Nesin et al, 2014, Proc Natl Acad Sci USA 111, 4197-4202). In analogy to the human disorders, mice either lacking Stim1 or Orai1, or carrying GoF mutations in these genes respectively recapitulate the main clinical signs of immunodeficiency or TAM/STRMK and represent valuable tools to investigate disease progression, uncover the underlying pathomechanisms, and identify therapeutic targets.

Most Orai1^{-/-} mice die perinatally, and the few surviving pups show defective B-cell and T-cell function and cytokine production, while heterozygous Orai^{+/-} animals are normal and fertile, demonstrating that a remaining Orai1 expression of 50% is sufficient to ensure vital SOCE activity (Gwak et al, 2008, Mol Cell Biol 28, 5209-5222).

Stim1^{R304W/+} mice harboring the most common TAM/STRMK mutation are smaller and weaker than their littermates, and manifest bone, platelet, spleen, and skin anomalies. Histological analyses of Stim1^{R304W/+} muscle sections revealed the presence of fibers with Ca²⁺ overload, and functional investigations in animals and on muscle extracts evidenced that the elevated cytosolic Ca²⁺ levels hamper regular muscle contraction and lead to sustained reticular stress, resulting in increased cell death and muscle fiber turnover (Silva-Rojas et al, 2019, Hum Mol Genet 28, 1579-1593; Silva-Rojas et al, 2021, Cells 10).

Despite this knowledge, there is currently no therapy for TAM/STRMK. According, there is a need to develop a therapy for the treatment of TAM and STRMK.

### SUMMARY OF THE INVENTION

The present invention relates to a therapy for the treatment of selected from the group consisting of tubular aggregate myopathy (TAM) and Stormorken syndrome (STRMK). In particular, this therapy uses a shRNA inhibiting the expression of ORAI1.

The inventors provide the evidence that the downregulation of the Ca²⁺ channel ORAI1 is able to antagonize disease development. This result is surprising and cannot be predicted for the following reasons.

Ca²⁺ entry is essential for the activation of the immune system in response to pathogens, and total loss of ORAI1 leads to immunodeficiency. In accordance, Orai1^{-/-} mice are perinatally lethal (Gwack et al., 2008, supra). In addition, heterozygous Orai1^{+/-} mice are normal and fertile, and heterozygous carriers of recessive ORAI1 mutations are healthy, indicating that the remaining ORAI1 expression of 50% is sufficient for normal physiology. Therefore, it remained to be determined if the permanent downregulation of ORAI1 by half is sufficient to at least partially improve the multi-systemic TAM/STRMK phenotype, and if acute ORAI1 downregulation by more than 50% procures an additional benefice without producing adverse effects.

TAM/STRMK patients and mice do not display increased ORAI1 expression levels (Silva-Rojas et al, 2019, supra; Hum Mol Genet 28, 1579-1593; Endo et al, 2015, supra), demonstrating that the excessive Ca²⁺ influx is not a consequence of an over-abundance of Ca²⁺ channels. It therefore remained to be determined if Ca²⁺ homeostasis can be rebalanced by Orai1 downregulation.

STIM1 mutants harboring TAM/STRMK mutations constitutively activate ORAI1. Downregulation of ORAI1 may therefore restrict Ca²⁺ influx, but it remained to be determined if the overall reduction of available ORAI1 channels will efficiently counteract the effect of overactive STIM1 and sufficiently reduce Ca²⁺ entry to normalize physiology.

ORAI1 has previously been identified as a potential target in other diseases and disorders, and most academic and private research teams use a pharmacological approach to screen for molecules occluding the channel. However, chemical compounds are usually unspecific, increasing the risk of undesirable side effects. An advantage of the therapy according to the present invention is that shRNA is specific of a given targeted mRNA. Another advantage of shRNA is the possibility to target a particular tissue through the promoter and the tropism of the vector virus. It however remained to be shown if the DNA-encoded shRNA is efficiently delivered, transcribed, and active, especially because shRNA design is often based on in silico predictions and because shRNA production and application involves technically challenging steps.

Taken together and based on the current scientific knowledge, shRNA-mediated downregulation of ORAI1 is an innovative therapeutic approach for TAM/STRMK. ORAI1 is not overexpressed in TAM/STRMK patients and mice, and total ORAI1 loss has been associated with severe immunodeficiency. Owing to its biological role as Ca²⁺ entry channel, the quest for pharmacological compounds blocking Ca² influx appears as the most obvious option, but is less target-specific than shRNA.

To provide the proof-of-concept that TAM/STRMK can efficiently be prevented by attenuating extracellular Ca²⁺ entry, the inventors crossed Stim1^{R304W/+} mice with Orai1^{+/-} mice expressing only 50% of the Ca²⁺ channel ORAI1, and the Stim1^{R430W/+}/Orai1^{+/-} offspring and control littermates underwent thorough phenotyping.

The Stim1^{R304W/+}Orai1^{-/-} mice showed a normal birth ratio and an improved postnatal weight gain compared with TAM/STRMK animals. The Stim1^{R304W+}/Orai1^{+/-} mice also showed a (statistically not significant) tendency of improved performances in hanging time, covered distance, and mean speed, as well as significantly ameliorated muscle contraction and relaxation kinetics and a normalized myofiber diameter. The skin, platelet, and spleen anomalies however remained unchanged. Overall, the reduction of Orai1 expression by half has no measurable negative impact on WT animals, and partially rescues the multi-systemic phenotype of TAM/STRMK mice.

Based on these results, the inventors intended to downregulate ORAI1 in postnatal tissues of Stim1^{R304W/+} mice through AAV-mediated shRNA delivery in order to provide a translational approach for prospective therapeutic trials in patients. They designed different shRNAs targeting both human and murine ORAI1 sequences and differing from ORAI2 and ORAI3, and they validated their efficacy and specificity in the cell model and in WT muscle. Finally, they performed intramuscular injections of TAM/STRMK mice and determined a significantly reduced Orai1 expression by up to 80% and a positive effect of two shRNAs on muscle function.

Abnormal Ca²⁺ balance has been associated with various rare and common disorders affecting skeletal muscle, heart, bones, brain, skin, or the immune and hormonal systems (Edwards et al, 2010, Cell Calcium 47, 458-467). The present approach targeting Orai1 expression with shRNAs to prevent and possibly revert TAM/STRMK might therefore also serve for the development of therapies for other Ca²⁺-related diseases.

Accordingly, the present invention relates to a short-hairpin RNA (shRNA), a nucleic acid encoding said shRNA or a pharmaceutical composition comprising it for use for the treatment of selected from the group consisting of tubular aggregate myopathy (TAM) and Stormorken syndrome (STRMK), said shRNA inhibiting the expression of ORAI1 (Calcium release-activated calcium channel protein 1). It further relates to the use of a shRNA, a nucleic acid encoding said shRNA or a pharmaceutical composition comprising it for the manufacture of a medicament for the treatment of selected from the group consisting of tubular aggregate myopathy (TAM) and Stormorken syndrome (STRMK), said shRNA inhibiting the expression of ORAI1. It also relates to a method for treating a disease selected from the group consisting of tubular aggregate myopathy (TAM) and Stormorken syndrome (STRMK) in a subject, comprising administering a therapeutically amount of a shRNA, a nucleic acid encoding said shRNA or a pharmaceutical composition comprising it to said subject, said shRNA inhibiting the expression of ORAI1, thereby improving muscle function.

In a first aspect, the shRNA specifically targets a sequence GATGAGCCTCAACGAGCA (SEQ ID NO: 5). More specifically, the nucleic acid encoding the shRNA comprises the sequence of SEQ ID NO: 6 (AGCTTTGTTTGGATGAGCCTCAACGAGCATTCAAGAGATGCTCGTTGAGGCTCATCCC-polyT) and/or the shRNA has the sequence of SEQ ID NO: 7 (GGATGAGCCTCAACGAGCATTCAAGAGATGCTCGTTGAGGCTCATCC).

In a second aspect, the shRNA specifically targets a sequence selected from CCCGAGTCACAGCAATCCGGA (SEQ ID NO: 1) and CCCGAGCCGCAGCAGTCCCGA (SEQ ID NO: 2). More specifically, the nucleic acid encoding the shRNA comprises the sequence of SEQ ID NO: 3 (AGCTTTGTTTGCCCGAGTCACAGCAATCCGGATTCAAGAGATCCGGATTGCTGTGACTCGGGCC-polyT) and/or the shRNA has the sequence of SEQ ID NO: 4 (GCCCGAGTCACAGCAATCCGGATTCAAGAGATCCGGATTGCTGTGACTCGGGC).

In a third aspect, the shRNA specifically targets a sequence selected in the group consisting of SEQ ID NOs: 48-156.

In additional, the present invention relates to a pharmaceutical composition comprising a shRNA or a nucleic acid encoding said shRNA and optionally a pharmaceutically acceptable carrier or excipient, wherein the shRNA inhibits the ORAI1 expression and specifically targets a sequence selected from CCCGAGTCACAGCAATCCGGA (SEQ ID NO: 1) and CCCGAGCCGCAGCAGTCCCGA (SEQ ID NO: 2). It relates to this pharmaceutical composition for use as a drug, to a shRNA or a nucleic acid encoding said shRNA as a medicament or to the use of a shRNA or a nucleic acid encoding said shRNA for the manufacture of a medicament, wherein the shRNA inhibits the ORAI1 expression and specifically targets a sequence selected from CCCGAGTCACAGCAATCCGGA (SEQ ID NO: 1) and CCCGAGCCGCAGCAGTCCCGA (SEQ ID NO: 2). More specifically, the nucleic acid encoding the shRNA comprises the sequence of SEQ ID NO: 3 (AGCTTTGTTTGCCCGAGTCACAGCAATCCGGATTCAAGAGATCCGGATTGCTGTGACTCGGGCC-polyT) and/or the shRNA has the sequence of SEQ ID NO: 4 (GCCCGAGTCACAGCAATCCGGATTCAAGAGATCCGGATTGCTGTGACTCGGGC).

Optionally, said nucleic acid encoding said shRNA is used as a naked DNA or is a vector encoding the shRNA, in particular a vector such as a viral vector, a plasmid or human artificial chromosomes (HAC).

For instance, the vector can be a viral vector such as adeno-associated vectors (AAV), adenoviral vectors, baculoviral vectors, herpes viral vectors, and retroviral vectors; especially lentiviral vectors. In a preferred aspect, the vector is an AAV vector.

Optionally, the nucleic acid encoding said shRNA is controlled by an ubiquitous promoter or a promoter specific for muscle.

### BRIEF DESCRIPTION OF THE FIGURES

FIG. 1. Normal Orai2 and Orai3 expression and increased body length in Stim1^{R304W/+}Orai1^{+/-} mice. (A) Crossing of Stim1^{R304W/+} and Orai1^{+/-} mice resulted in four genotypes: WT, Orai1^{+/-}, Stim1^{R304W/+} and Stim1^{R304W/+}Orai1^{+/-}. (B-D) Orai1 expression was reduced by half in Stim1^{R304W/+}Orai1^{+/-} offspring, while Orai2 and Orai3 expression was unaffected (n=4-6). (E) Stim1^{R304W/+}Orai1^{+/-} mice were larger than the Stim1^{R304W/+} littermates at 4 months (n=5-9). (F-G) The gastrocnemius was hyotrophic in both Stim1^{R304W/+} and Stim1^{R304W/+}Orai1^{+/-} mice, while the soleus hypertrophy was partially rescued in Stim1^{R304W/+}Orai1^{+/-} mice at 4 months. (H) Statistically indistinguishable fat layer diameter in WT, Orai1^{+/-}, Stim1^{R304W/+}, and Stim1^{R304W/+}Orai1^{+/-} skin samples at 4-8 months of age, but improvement in single Stim1^{R304W/+}Orai1^{+/-} animals (n=6-11). (I) Increased coagulation time in Stim1^{R304W/+} and Stim1^{R304W/+}Orai1^{+/-} mice at 2 months compared to healthy controls (n=6-11). Graphs represent mean±SEM. Significant differences are indicated as */α/$ P<0.05, **/αα/$$ P<0.01, ***/ααα/$$$ P<0.001, and ****/αααα/$$$$ P<0.0001 with * reflecting the comparison with the WT group, a the comparison with the Orai1^{+/-} group, and $ for the comparison with the Stim1^{R304W/+}Orai1^{+/-} group.

FIG. 2. Improved weight gain and bone structure in Stim1^{R304W/+}Orai1^{+/-} mice. (A) Body weight evolution was ameliorated in Stim1^{R304W/+}Orai1^{+/-} mice compared with Stim1^{R304W/+} littermates over the first months of life (n=11-17). (B) 3D reconstruction of the femur microarchitecture illustrated a similar trabecular density in Stim1^{R304W/+}Orai1^{+/-} mice and healthy WT and Orai1^{+/-} controls. (C) Histological H&E staining of back skin sections at 8 months evidenced a normalized fat layer thickness (arrows) only in single Stim1^{R304W/+}Orai1^{+/-} mice. (D-F) Relative spleen weight, megakaryocyte numbers, and platelet counts were comparable in Stim1^{R304W/+} and Stim1^{R304W/+}Orai1^{+/-} mice and significantly differed from the healthy controls (n=5-9). Graphs represent mean±SEM. Significant differences are indicated as */α/$ P<0.05, **/αα/$$ P<0.01, ***/ααα/$$$ P<0.001, and ****/αααα/$$$$ P<0.0001 with * reflecting the comparison with the WT group, a the comparison with the Orai1^{+/-} group, and $ for the comparison with the Stim1^{R304W/+}Orai1^{+/-} group.

FIG. 3. Partially improved muscle performance of Stim1^{R304W/+}Orai1^{+/-} mice. (A) Stim1^{R304W/+}Orai1^{+/-} mice showed a continuous but not significant tendency of increased hanging times compared with Stim1^{R304W/+} littermates between 1 and 4 months (n=11-17). (B) The velocity of Stim1^{R304W/+}Orai1^{+/-} mice in the open field arena was indistinguishable from WT and Orai1^{+/-} controls at 10 weeks of age and slightly but not significantly improved compared with Stim1^{R304W/+} littermates (n=7-14). (C) In situ measurements at 2 months revealed a slightly but not significantly elevated muscle force of Stim1^{R304W/+}Orai1^{+/-} mice compared with Stim1^{R304W/+} littermates (n=5-7). (D-F) Stimulation frequencies of 1-20 Hz evidenced a significant shift of the Stim1^{R304W/+}Orai1^{+/-} muscle contraction properties towards normal values, while muscle relaxation following tetanic stimulation was similar in Stim1^{R304W/+} and Stim1^{R304W/+}Orai1^{+/-} mice (n=5-9). Graphs represent mean±SEM. Significant differences are indicated as */α/$ P<0.05, **/αα/$$ P<0.01, ***/ααα/$$$ P<0.001, and ****/αααα/$$$$ P<0.0001 with * reflecting the comparison with the WT group, a the comparison with the Orai1^{+/-} group, and $ for the comparison with the Stim1^{R304W/+}Orai1^{+/-} group.

FIG. 4. Grip strength, open field test, and muscle force of Stim1^{R304W/+}Orai1^{+/-} mice. (A-C) Non-significant tendencies of increased of 4-paw grip strength, covered distance in the open field arena, and maximal muscle force of Stim1^{R304W/+}Orai1^{+/-} mice compared with Stim1^{R304W/+} controls at 4 months and weeks of age (n=5-14). (C) Graphs represent mean±SEM. Significant differences are indicated as ***/ααα P<0.001, and ****/αααα P<0.0001 with * reflecting the comparison with the WT group and α the comparison with the Orai1^{+/-} group.

FIG. 5. Improved muscle contraction properties in Stim1^{R304W/+}Orai1^{+/-} mice. (A-C) Significantly faster muscle contraction and non-significant tendency of improved muscle relaxation following single stimulations in Stim1^{R304W/+}Orai1^{+/-} compared with Stim1^{R304W/+} mice at 4 months (n=5-9). (D-E) Specific and normalized force produced across 80 stimulation trains of 40 Hz illustrate different fatigue curves of Stim1^{R304W/+} and Stim1^{R304W/+}Orai1^{+/-} muscle compared with healthy controls at 4 months (n=5-9). (F) Quantification of fatigue as the cumulation of force following 80 stimulations (n=5-9). (G) Quantification of fatigue as the ratio between the last and the highest force level within the stimulation train (n=5-9). Graphs represent mean±SEM. Significant differences are indicated as */α/$ P<0.05, **/αα/$$ P<0.01, ***/ααα/$$$ P<0.001, and ****/αααα/$$$$ P<0.0001 with * reflecting the comparison with the WT group, α the comparison with the Orai1^{+/-} group, and $ for the comparison with the Stim1^{R304W/+}Orai1^{+/-} group.

FIG. 6. Increased myofiber size and improved autophagic flux in Stim1^{R304W/+}Orai1^{+/-} mice. (A) H&E staining on muscle sections from both Stim1^{R304W/+} and Stim1^{R304W/+}Orai1^{+/-} mice at 4 months revealed signs of muscle fiber degeneration such as centralized nuclei (blue arrows), regenerating fibers (green arrow) and immune cell infiltrations (black arrows). (B-C) Different fiber size distribution in Stim1^{R304W/+} and Stim1^{R304W/+}Orai1^{+/-} mice and normalization of fibers with a MinFeret diameter of >40 μm in Stim1^{R304W/+}Orai^{+/-} muscle at 4 months (n=5). (D-F) Quantification revealed a comparable number of fibers with centralized nuclei and similar expression levels of the UPR markers Hspa5 and Hsp90b1 in Stim1^{R304W/+} and Stim1^{R304W/+}Orai1^{+/-} muscle at 4 months (n=5-6). (G) Increased LC3-II and p62 protein levels in Stim1^{R304W/+} muscle samples compared to WT at 4 months (n=6). (H-I) Reduced LC3-II and normalized p62 protein levels in Stim1^{R304W/+}Orai1^{+/-} muscle samples compared to Stim1^{R304W/+} mice at 4 months (n=5-6). Graphs represent mean±SEM. Significant differences are indicated as */α/$ P<0.05, **/αα/$$ P<0.01, ***/ααα/$$$ P<0.001, and ****/αααα/$$$$ P<0.0001 with * reflecting the comparison with the WT group, α the comparison with the Orai1^{+/-} group, and $ for the comparison with the Stim1^{R304W/+}Orai1^{+/-} group.

FIG. 7. Partially resolved autophagy defects in Stim1^{R304W/+}Orai^{+/-} muscle. (A) Slight decrease of Map1lc3a, Map1lc3b (both encoding LCO-II), and Sqstm1 (p62) expression in Stim1^{R304W/+} muscle compared with the WT at 4 months (n=4-5). (B-D) Western blots on muscle extracts evidences intense LC3-II and p62 signals in Stim1^{R304W/+} muscle and partial normalization in Stim1^{R304W/+}Orai1^{+/-} muscle (n=5-6). Ponceau S staining served as loading control. Graph represents mean±SEM. Significant differences are indicated as * P<0.05, with * reflecting the comparison with the WT group.

FIG. 8. In cellulo and in vivo validation of shRNAs. Schematic representation of Orai1 mRNA and positions targeted by the shRNAs. sh22, sh190 and sh760 efficiently reduced Orai1 expression in transfected C2C12 cells, while scrambles 2 and 3 (Scr2, Scr3) had no effect (n=4). The dashed line reflects the Orai1 expression level in untreated cells. AAV9 particles containing the shRNAs were injected into the tibialis anterior of 1-month old mice. sh22 and sh190 yielded a reduction of Orai1 expression of 80% compared with scramble shRNAs and NaCl treatment (dashed line) 4 weeks post injection (n=6-7). Graphs represent mean±SEM. Significant differences are indicated as *** P<0.001 and **** P<0.0001 with * reflecting the comparison with the scramble-injected group.

FIG. 9. Improved muscle contraction and relaxation properties in TAM/STRMK mice through Orai1 silencing 2 months post shRNA injection. (A) sh22 (top) and sh190 (bottom) yielded 80% decrease of Orai1 expression in Stim1^{R304W/+} muscle compared to scramble-injected WT, NaCl-injected WT (black dashed line), and NaCl-injected Stim1^{R304W/+} (red dashed line) controls (n=4-6). (B) Shifted force production towards normal values at low stimulation frequencies in Stim1^{R304W/+} tibialis anterior treated with sh22 (top) and sh190 (bottom) compared with scramble-injected controls (n=4-8). (C-D) Improved muscle relaxation after single and tetanic stimulation of Stim1^{R304W/+} tibialis anterior injected with sh22 (top) and sh190 (bottom) compared with scramble-injected controls (n=3-8). (E) The time required for a muscle relaxation of 70% is significantly reduced in Stim1^{R304W/+} tibialis anterior injected with sh22 (top) and sh190 (bottom) compared with scramble-injected controls, NaCl-injected WT (black dashed line), and NaCl-injected Stim1^{R304W/+} mice (red dashed line) (n=3-8). Graphs represent mean±SEM. Significant differences are indicated as */α/$ P<0.05, **/αα/$$ P<0.01, ***/ααα/$$$ P<0.001 and ****/αααα/$$$$ P<0.0001 with * reflecting the comparison with the scramble-injected WT group, α the comparison with the shRNA-injected WT sh group, and $ for the comparison with the scramble-injected Stim1^{R304W/+} group.

FIG. 10. Normal Orai2 and Orai3 expression following shRNA treatment. (A-D) Comparable Orai2 and Orai3 expression levels in Stim1^{R304W/+} muscle treated with Orai1-specific shRNAs, scramble shRNAs or NaCl 2 months post injection (n=4-7). Graphs represent mean±SEM.

FIG. 11. No effect of sh22 and sh190 on fiber size and muscle degeneration. (A-D) The low percentage of big fibers and the increased proportion of myofibers with central nuclei in Stim1^{R304W/+} muscle was not rescued by shRNA treatment 2 months post injection (n=3-7). Graphs represent mean±SEM. Significant differences are indicated as */α P<0.05 and **/αα P<0.01 with * reflecting the comparison with the scramble-injected WT group and α the comparison with the shRNA-injected WT group.

FIG. 12. No effect of sh22 and sh190 on UPR and autophagosome accumulation. (A-B) Comparable expression of the UPR marker Hsp5 in treated and untreated Stim1^{R304W/+} muscle 2 months post injection (n=3-7). (C-D) Comparable protein levels of lipidated LC3 (LC3-II) and p62 in treated and untreated Stim1^{R304W/+} muscle 2 months post injection (n=4). Graphs represent mean±SEM. Significant differences are indicated as */α P<0.05, **/αα P<0.01, and ***/ααα/$$$ P<0.0001 with * reflecting the comparison with the scramble-injected WT group and α the comparison with the shRNA-injected WT group. FIG. 13. LC3-II and p62 protein levels. Western blots indicated comparable LC3-II and p62 signal intensities in extracts from treated and untreated Stim1^{R304W/+} muscles 2 months post injection (n=4). Ponceau S staining served as loading control.

### DETAILED DESCRIPTION OF THE INVENTION

The present invention relates to a shRNA inhibiting the expression of ORAI1 (Calcium release-activated calcium channel protein 1), a nucleic acid encoding said shRNA, or a pharmaceutical composition comprising the shRNA or the nucleic acid encoding said shRNA for use for the treatment of a disease selected from the group consisting of tubular aggregate myopathy (TAM) and Stormorken syndrome (STRMK). It further relates to the use of a shRNA, a nucleic acid encoding said shRNA or a pharmaceutical composition comprising it for the manufacture of a medicament for the treatment of a disease selected from the group consisting of tubular aggregate myopathy (TAM) and Stormorken syndrome (STRMK), said shRNA inhibiting the expression of ORAI1. It also relates to a method for treating a disease selected from the group consisting of tubular aggregate myopathy (TAM) and Stormorken syndrome (STRMK) in a subject, comprising administering a therapeutically amount of a shRNA, a nucleic acid encoding said shRNA or a pharmaceutical composition comprising it to said subject, said shRNA inhibiting the expression of ORAI1, thereby improving muscle function.

ORAI1 is Calcium release-activated calcium channel protein 1. ORAI1 is described in UniProt under Q96D31 and in HGNC under 25896. The amino acid sequence is disclosed in NP_116179.2 and the nucleotide sequence encoding it is disclosed in NM_032790.3.

ORAI2 is protein orai-2. ORAI2 is described in UniProt under Q96SN7 and in HGNC under 21667. The amino acid sequence is disclosed in NP_001119812, NP_001258747, NP_001258748, and NP_116220 and the nucleotide sequence encoding it is disclosed in NM_032831, NM_001126340, NM_001271818 and NM_001271819.

ORAI3 is protein orai-3. ORAI3 is described in UniProt under Q9BRQ5 and in HGNC under 28185. The amino acid sequence is disclosed in NP_689501.1 and the nucleotide sequence encoding it is disclosed in NM_152288.2.

STIM1 is Stromal interaction molecule 1. Human STIM1 is described in UniProt under Q13586, in HGNC under 11386, STIM1 includes a signal peptide at position 1-22, a N terminal domain at positions 23-213, a transmembrane domain at positions 214-234 and a C-terminal domain at positions 235-685, as disclosed in the canonical sequence of STIM1 Uniprot Q13586-1, STIM1-S. The amino acid sequence is disclosed in NP_003147.2 and the nucleotide sequence encoding it is disclosed in NM_003156.3. A longer isoform (STIM1L) is disclosed under Uniprot GOXQ39. The amino acid sequence of STIM1L is disclosed in NP_001264890 and the nucleotide sequence encoding it is disclosed in NM_001277961.

Preferably, the shRNA is specific for ORAI1. More particularly, the expression of ORAI1 is decreased by the shRNA by at least 50%, 60%, 70%, 80% or 90% compared to the ORAI1 expression in absence of any shRNA. It does not significantly inhibit the expression of ORAI2 and ORAI3. In particular, the expression of ORAI2 and ORAI3 is decreased by the shRNA at most of 20%, preferably at most of 10%, still more preferably at most of 5% compared to the ORAI2/3 expression, respectively, in absence of any shRNA. The inhibition of the expression by the shRNA can be measured by any method known by the skilled person. In particular, the inhibition of the expression by the shRNA can be measured by the method detailed in section “shRNA screening and intramuscular AAV injection” of the example section either by cell shRNA screening or by intramuscular AAV screening.

Short RNAs can be introduced into the cell as either short hairpin RNAs (shRNAs). In mammalian cells, shRNAs are at least 10, 15 or 20 base pair (bp) long, typically 19, 20, 21, 22, 23, 24 or 25 bp long, and are designed to have complementarity to the target sequence. shRNAs are double stranded RNAs (dsRNAs) that contain a loop structure, and are processed into siRNA by the host enzyme DICER, an endo-RNase that contains RNase 11 domains (Bernstein, Caudy et al. 2001). More particularly, shRNA generally consists of two complementary 19-22 bp RNA sequences linked by a short loop of 4-11 nucleotides.

As used therein, the term “shRNA” refers to small hairpin RNA or short hairpin RNA and is used to silence genes via RNA interference. Usually, the shRNA may be introduced into the target cell using a vector or as naked DNA. The shRNA hairpin structure is cleaved by other substances in the cell to become siRNA. The design of shRNA is well-known by the person skilled in the art. For illustration or reference, for instance please see the review Bofill-De-Ros et al 2016, Methods, 103, 157-166; Pelossof et al., Nature Biotechnology. 2017 April; 35(4):350-353.

The shRNA has a target sequence with ORAI1, especially within ORAI1 coding sequence. Optionally, the target sequence of the shRNA is located in exon 1 or exon 2 of ORAI1. Preferably, the target sequence of the shRNA is located in exon 1 of ORAI1.

Optionally, the target sequence of the shRNA can be selected to be conserved among human and an animal model, especially mouse.

Optionally, the target sequence can be selected from the group consisting of SEQ ID NOs: 1, 2, 5 and 48-156. In a particular aspect, the target sequence is selected from the group consisting of SEQ ID NOs: 1, 2 and 5. In a first preferred aspect, the target sequence is SEQ ID NO: 1 or SEQ ID NO: 2. In a second preferred aspect, the target sequence is SEQ ID NO: 5.

In this first preferred aspect, the shRNA may have the sequence of SEQ ID NO: 4. Optionally, the shRNA of SEQ ID NO: 4 may comprised one to four substitutions of one nucleotide. The present invention also relates to an nucleic acid encoding such a shRNA. In particular, this nucleic acid comprises the sequence of SEQ ID NO: 3. By poly-T, is intended to refer to several T nucleotides, for instance from 4 T to 10T, in particular 5T. In a very specific aspect, such nucleic acids encoding the shRNA are disclosed in SEQ ID NOs 34 and 35.

The present invention relates to a shRNA with a target sequence of SEQ ID NO: 1 or SEQ ID NO: 2. It relates to a shRNA of SEQ ID NO: 4, optionally with one to four substitutions of one nucleotide. It relates to a nucleic acid encoding a shRNA with a target sequence of SEQ ID NO: 1 or SEQ ID NO: 2 or comprising or consisting of the sequence of SEQ ID NO: 4, such as the sequence of SEQ ID NO: 3 or of SEQ ID NOs 34 and 35. It also specifically relates to a pharmaceutical composition comprising a shRNA with a target sequence of SEQ ID NO: 1 or SEQ ID NO: 2, a shRNA comprising or consisting of the sequence of SEQ ID NO: 4, or a nucleic acid encoding a shRNA with a target sequence of SEQ ID NO: 1 or SEQ ID NO: 2 or comprising or consisting of the sequence of SEQ ID NO: 4, such as the sequence of SEQ ID NO: 3 or of SEQ ID NOs 34 and 35. It relates to this pharmaceutical composition for use as a medicament or drug, to the use of this pharmaceutical composition for the manufacture of a medicament, or to a method for treating a disease or disorder in a subject in need thereof, comprising administering a therapeutically effective amount of this pharmaceutical composition.

In this second preferred aspect, the shRNA may have the sequence of SEQ ID NO: 7. The present invention also relates to an nucleic acid encoding such a shRNA. In particular, this nucleic acid comprises the sequence of SEQ ID NO: 6. In a very specific aspect, such nucleic acids encoding the shRNA are disclosed in SEQ ID NOs 38 and 39. By poly-T, is intended to refer to several T nucleotides, for instance from 4 T to 10T, in particular 5T.

The present invention further relates to the use of a pharmaceutical composition comprising a shRNA as described above.

It is relates to the use of a nucleic acid encoding a shRNA as described above. It also relates to a pharmaceutical composition comprising a nucleic acid encoding a shRNA and its use.

The nucleic acid encoding a shRNA further comprises all elements necessary for its expression in a human cell, these elements being operatively linked to the sequence encoding the shRNA. The nucleic acid encoding a shRNA comprises sufficient cis-acting elements for expression; other elements for expression can be supplied by the host cell or in an in vitro expression system. As used herein, “operably linked” sequences include both expression control sequences that are contiguous with the sequence encoding the shRNA and expression control sequences that act in trans or at a distance to control the gene of interest. Expression control sequences include appropriate transcription initiation, termination, promoter and enhancer sequences. A great number of expression control sequences, including promoters which are native, constitutive, inducible and/or tissue-specific, are known in the art and may be utilized. Additional promoter elements, e.g., enhancers, regulate the frequency of transcriptional initiation.

One of these elements is a promoter. The promoter can be ubiquitous or specific for a tissue or a cellular type, in particular muscle-specific. In particular, the promoter can be specific for muscle. In another particular aspect, the promoter is inducible, thereby allowing to control its expression and render it specific of a tissue, especially specific for muscle. Alternatively, the promoter can be a constitutive promoter. Typical promoters used for controlling shRNA expression are RNA pol Ill or modified pol II promoters such as U6, H1 or 7S K promoters. The nucleic acid may also include an enhancer sequence and other regulatory elements which are required for expression of the gene product, for instance regulatory elements that can influence the tissue specificity of the expression or that can modulate the level of expression.

The nucleic acid encoding the shRNA can be used as a naked DNA or can be included into a vector. The vector can be for instance a cosmid, a plasmid (e.g., naked or contained in liposomes), a viral vector or human artificial chromosomes (HAC). Thus, the term “vector” includes an autonomously replicating plasmid or a virus. The viral vector can be an adeno-associated vectors (AAV), adenoviral vectors, baculoviral vectors, herpes viral vectors, and retroviral vectors; especially lentiviral vectors. In a preferred aspect, the vector is an adeno-associated vectors (AAV). Preferably, the vector can be selected for having a tropism for a particular tissue, especially with a tropism for muscle.

For example, vectors derived from retroviruses such as the lentivirus are suitable tools to achieve long-term gene transfer since they allow long-term, stable integration of a transgene and its propagation in daughter cells.

In a preferred aspect, the vector is an adeno-associated virus (AAV). Adeno-associated viral (AAV) vectors have become powerful gene delivery tools for the treatment of various disorders. AAV vectors possess a number of features that render them ideally suited for gene therapy, including a lack of pathogenicity, minimal immunogenicity, and the ability to transduce postmitotic cells in a stable and efficient manner. Expression of a particular gene contained within an AAV vector can be specifically targeted to one or more types of cells by choosing the appropriate combination of AAV serotype, promoter, and delivery method.

More than 30 naturally occurring serotypes of AAV are available. Many natural variants in the AAV capsid exist, allowing identification and use of an AAV with properties specifically suited for skeletal muscle. AAV viruses may be engineered using conventional molecular biology techniques, making it possible to optimize these particles for cell specific delivery of myotubularin nucleic acid sequences, for minimizing immunogenicity, for tuning stability and particle lifetime, for efficient degradation, for accurate delivery to the nucleus, etc.

Among the serotypes of AAVs isolated from human or non-human primates (NHP) and well characterized, human serotype 2 is the first AAV that was developed as a gene transfer vector; it has been widely used for efficient gene transfer experiments in different target tissues and animal models. Clinical trials of the experimental application of AAV2 based vectors to some human disease models are in progress, especially AAV2.5. Other useful AAV serotypes include AAV1, AAV3, AAV4, AAV5, AAV6, AAV7, AAV8, AAV9, AAV10, as well as AAV-DJ and AAV-PHP.S. In a particular aspect, the AAV could be selected from the group consisting of AAV1, AAV2.5, AAV2.6, AAV2.8, AAVrh8, AAV2.9 and AAVrh74

In one aspect, the vectors contain, at a minimum, sequences encoding a selected AAV serotype capsid, e.g., an AAV8 capsid, or a fragment thereof. In another aspect, useful vectors contain, at a minimum, sequences encoding a selected AAV serotype rep protein, e.g., AAV8 rep protein, or a fragment thereof. Optionally, such vectors may contain both AAV cap and rep proteins.

The AAV vectors may further contain a minigene comprising a nucleic acid sequence encoding the shRNA as described above which is flanked by AAV 5′ (inverted terminal repeat) ITR and AAV 3′ ITR. A suitable recombinant adeno-associated virus (AAV) is generated by culturing a host cell which contains a nucleic acid sequence encoding an adeno-associated virus (AAV) serotype capsid protein, or fragment thereof, as defined herein; a functional rep gene; a minigene composed of, at a minimum, AAV inverted terminal repeats (ITRs) and a nucleic acid sequence encoding the shRNA as described above; and sufficient helper functions to permit packaging of the minigene into the AAV capsid protein. The components required to be cultured in the host cell to package an AAV minigene in an AAV capsid may be provided to the host cell in trans. Alternatively, any one or more of the required components (e.g., minigene, rep sequences, cap sequences, and/or helper functions) may be provided by a stable host cell which has been engineered to contain one or more of the required components using methods known to those of skill in the art.

The minigene, rep sequences, cap sequences, and helper functions required for producing the rAAV of the invention may be delivered to the packaging host cell in the form of any genetic element which transfers the sequences carried thereon. The selected genetic element may be delivered using any suitable method, including those described herein and any others available in the art. The methods used to construct any embodiment of this invention are known to those with skill in nucleic acid manipulation and include genetic engineering, recombinant engineering, and synthetic techniques. Similarly, methods of generating rAAV virions are well known and the selection of a suitable method is not a limitation on the present invention.

Unless otherwise specified, the AAV ITRs, and other selected AAV components described herein, may be readily selected from among any AAV serotype, including, without limitation, AAV1, AAV2, AAV3, AAV4, AAV5, AAV6, AAV7, AAV8, AAV9, AAV10, as well as AAV-DJ and AAV-PHP.S or other known or as yet unknown AAV serotypes. These ITRs or other AAV components may be readily isolated from an AAV serotype using techniques available to those of skill in the art. Such an AAV may be isolated or obtained from academic, commercial, or public sources (e.g., the American Type Culture Collection, Manassas, Va.). Alternatively, the AAV sequences may be obtained through synthetic or other suitable means by reference to published sequences such as are available in the literature or in databases such as, e.g., GenBank, PubMed, or the like.

The minigene is composed of, at a minimum, a nucleic acid sequence encoding the shRNA as described above and its regulatory sequences, and 5′ and 3′ AAV inverted terminal repeats (ITRs). In one aspect, the ITRs of AAV serotype 2 are used. However, ITRs from other suitable serotypes may be selected. It is this minigene which is packaged into a capsid protein and delivered to a selected host cell. The nucleic acid sequence encoding the shRNA as described above is operatively linked to regulatory components in a manner which permits transgene transcription and/or expression in a host cell.

In addition to the major elements identified above for the minigene, the AAV vector generally includes conventional control elements which are operably linked to the transgene in a manner which permits its transcription and/or expression in a cell transfected with the plasmid vector or infected with the virus produced by the invention.

In one aspect, a naked nucleic acid encoding the shRNA or naked shRNA can be used. It is well known in the art that the use of naked isolated nucleic acid structures, including for example naked DNA, works well with inducing expression in muscle. As such, the present invention encompasses the use of such compositions for local delivery to the muscle and for systemic administration (Wu et al., 2005, Gene Ther, 12(6): 477-486). The nucleic acid encoding the shRNA or shRNA may be administered in free (naked) form or by the use of delivery systems that enhance stability and/or targeting, e.g., liposomes, or incorporated into other vehicles, such as hydrogels, cyclodextrins, biodegradable nanocapsules, bioadhesive microspheres, or proteinaceous vectors, or in combination with a cationic peptide. They can also be coupled to a biomimetic cell penetrating peptide. They may also be administered in the form of their precursors or encoding DNAs.

In the case where a non-viral delivery system is utilized, an exemplary delivery vehicle is a liposome. The use of lipid formulations is contemplated for the introduction of the nucleic acids into a host cell (in vitro, ex vivo or in vivo). In another aspect, the nucleic acid may be associated with a lipid. The nucleic acid associated with a lipid may be encapsulated in the aqueous interior of a liposome, interspersed within the lipid bilayer of a liposome, attached to a liposome via a linking molecule that is associated with both the liposome and the oligonucleotide, entrapped in a liposome, complexed with a liposome, dispersed in a solution containing a lipid, mixed with a lipid, combined with a lipid, contained as a suspension in a lipid, contained or complexed with a micelle, or otherwise associated with a lipid. Lipid, lipid/DNA or lipid/expression vector associated compositions are not limited to any particular structure in solution.

The present invention relates to a pharmaceutical composition comprising a shRNA or a nucleic acid encoding a shRNA.

The pharmaceutical composition of the invention is formulated in accordance with standard pharmaceutical practice (see, e.g., Remington: The Science and Practice of Pharmacy (20th ed.), ed. A. R. Gennaro, Lippincott Williams & Wilkins, 2000 and Encyclopedia of Pharmaceutical Technology, eds. J. Swarbrick and J. C. Boylan, 1988-1999, Marcel Dekker, New York) known by a person skilled in the art.

Possible pharmaceutical compositions include those suitable for oral, rectal, intravaginal, mucosal, topical (including transdermal, buccal and sublingual), or parenteral (including subcutaneous (sc), intramuscular (im), intravenous (iv), intra-arterial, intradermal, intrasternal, injection, or infusion techniques) administration. For these formulations, conventional excipient can be used according to techniques well known by those skilled in the art.

In particular, intramuscular or systemic administration is preferred. More particularly, in order to provide a localized therapeutic effect, specific muscular or intramuscular administration routes are preferred.

Pharmaceutical compositions according to the invention may be formulated to release the active drug substantially immediately upon administration or at any predetermined time or time period after administration.

The present invention relates to a method for treating a disease selected from the group consisting of tubular aggregate myopathy (TAM) and Stormorken syndrome (STRMK) in a subject suffering of said disease and having a susceptibility to develop said disease, comprising administering a therapeutically amount of a shRNA, a nucleic acid encoding said shRNA or a pharmaceutical composition comprising it to said subject, thereby improving a symptom of the disease.

In a particular aspect, the subject to be treated presents mutations in the STIM1 gene, for instance the substitution selected from the group consisting of H72Q, N80T, D84G, H109N, H109R, 1115F and R304W.

As used herein, “treating a disease or disorder”, especially “treating a disease selected from the group consisting of tubular aggregate myopathy (TAM) and Stormorken syndrome (STRMK)” means reducing the frequency with which a symptom of the disease or disorder is experienced by a patient. Disease and disorder are used interchangeably herein. To “treat” a disease as the term is used herein, means to reduce the frequency or severity of at least one sign or symptom of a disease or disorder experienced by a subject. Within the context of the invention, the term treatment denotes curative, symptomatic, and preventive treatment. As used herein, the term “treatment” of a disease refers to any act intended to extend life span of subjects (or patients) such as therapy and retardation of the disease progression. The treatment can be designed to eradicate the disease, to stop the progression of the disease, and/or to promote the regression of the disease. The term “treatment” of a disease also refers to any act intended to decrease the symptoms associated with the disease. More specifically, the treatment according to the invention is intended to delay the appearance of or revert phenotypes or symptoms of the disease, ameliorate the motor and/or muscular behavior and/or lifespan. More particularly, the symptom can be selected from the group consisting of childhood-onset muscle weakness with miosis, ichthyosis, short stature, hyposplenism, thrombocytopenia, and dyslexia. In a preferred aspect, the treatment improves muscle function.

A disease or disorder is “alleviated” if the severity of a symptom of the disease or disorder, the frequency with which such a symptom is experienced by a patient, or both, is reduced. A “therapeutic” treatment is a treatment administered to a subject who exhibits signs of pathology, for the purpose of diminishing or eliminating at least one or all of those signs.

The phrase “therapeutically effective amount,” as used herein, refers to an amount that is sufficient or effective to prevent or treat (delay or prevent the onset of, prevent the progression of, inhibit, decrease or reverse) a disease or disorder, including provision of a beneficial effect to the subject or alleviating symptoms of such diseases.

The terms “patient,” “subject,” “individual,” and the like are used interchangeably herein, and refer to any animal, or cells thereof whether in vitro or in situ, amenable to the methods described herein. In certain non-limiting embodiments, the patient, subject or individual is a human. Preferably the subject is a human patient whatever its age or sex. Embryos, fetuses, new-borns (neonates), infants, children/adolescents are included as well. In the context of the present invention, patients can be typically divided into neonates, children/adolescents and adults, as they display a different severity of the disease; the earlier the onset, the more severe the disease is. Embryos and fetuses refer to unborn offspring; neonates typically encompass newborns from day 0 to about 1 year old, while childhood/adolescents can range from about 1-2 years old patients to about 16 years-old patients (included). Adults may accordingly comprise those aged over 16 years old.

In a particular aspect, the therapeutically effective amount to be administered according to the invention is an amount sufficient to alleviate at least one or all of the symptom of the disease, or to improve muscle function of subject with such a disease. The amount to be administered can be determined by standard procedure well known by those of ordinary skill in the art. Physiological data of the patient (e.g. age, size, and weight), the routes of administration and the disease to be treated have to be taken into account to determine the appropriate dosage. One skilled in the art will recognize that the amount to be administered will be an amount that is sufficient to treat at least one or all of the symptoms of the disease, or to improve muscle function of subject with such a disease. Such an amount may vary inter alia depending on the gender, age, weight, overall physical condition of the patient, etc. and may be determined on a case by case basis. The amount may also vary according to other components of a treatment protocol (e.g. administration of other pharmaceuticals, etc.). Generally, when the therapeutic agent is a nucleic acid, a suitable dose is in the range of from about 1 mg/kg to about 100 mg/kg, and more usually from about 2 mg/kg/day to about 10 mg/kg. If a viral-based delivery of the nucleic acid is chosen, suitable doses will depend on different factors such as the virus that is employed, the route of delivery (intramuscular, intravenous, intra-arterial or other), but may typically range from 10-9 to 10-15 viral particles/kg. Those of skill in the art will recognize that such parameters are normally worked out during clinical trials. Further, those of skill in the art will recognize that, while disease symptoms may be completely alleviated by the treatments described herein, this need not be the case. Even a partial or intermittent relief of symptoms may be of great benefit to the recipient. In addition, treatment of the patient may be a single event, or the patient is administered with the pharmaceutical composition on multiple occasions, that may be, depending on the results obtained, several days apart, several weeks apart, or several months apart, or even several years apart.

### EXAMPLES

Mice harboring the most common TAM/STRMK mutation STIM1 p.Arg304Trp (R304W) were shown to recapitulate the main clinical signs of the human disorder, and the availability offers the possibility to establish and validate therapeutic approaches. In order to antagonize the development of TAM/STRMK, the inventors crossed Stim1^{R304W/+} mice (Silva-Rojas, R. et al., Hum Mol Genet 28, 1579-1593, 2019) with Orai1^{+/-} animals (Gwack, Y. et al., Mol Cell Biol 28, 5209-5222, 2008) expressing 50% of the Ca²⁺ channel ORAI1. The resulting WT, Orai1^{+/-}, Stim1^{R304W/+}, and Stim1^{R304W/+}Orai1^{+/-} offspring underwent comparative phenotyping to assess birth ratio, muscle force, platelet number, bone morphology, as well as skin and spleen histology to conclude on the therapeutic potential of Orai1 downregulation.

### Example 1: Normalized Birth Ratio, and Improved Body Size and Weight Gain of Stim1^{R304W/+}Orai1^{+/-} Mice

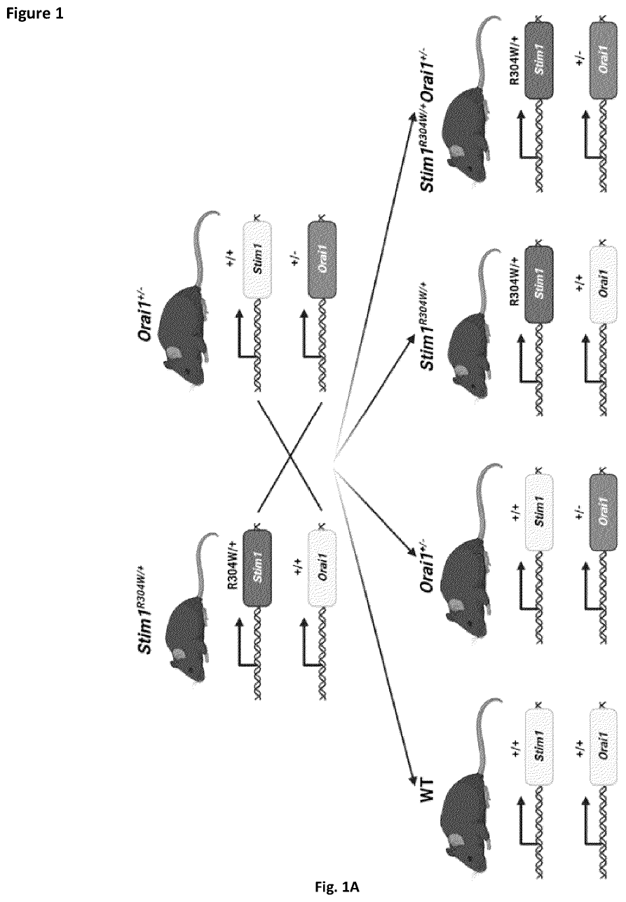

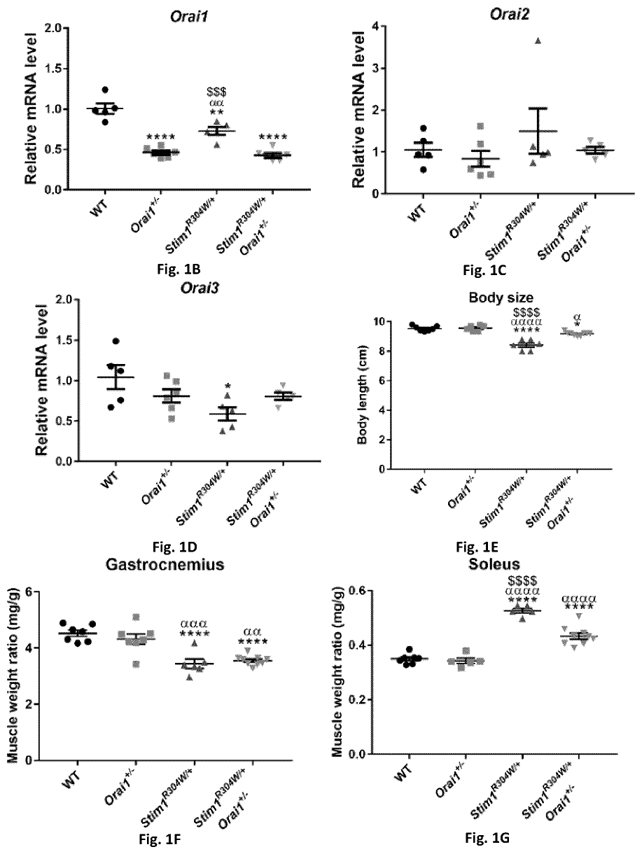

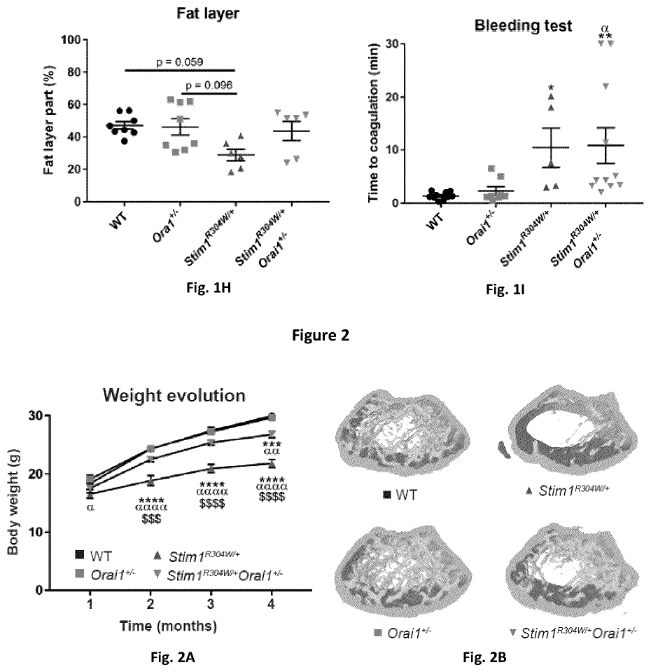

It was previously reported that the number of Stim1^{R304W/+} pups is below the expected Mendelian ratio and that the viable animals are smaller than their WT littermates throughout life (Silva-Rojas, R. et al., Hum Mol Genet 28, 1579-1593, 2019), pointing to a crucial role of SOCE for prenatal and postnatal development. To assess whether Orai1 downregulation prevents sporadic embryonic death and improves early growth stages, the inventors crossed Stim1^{R304W/+} with Orai1^{+/-} mice and genotyped almost 300 offspring seven days after birth (FIG. 1A). In line with WT (23%) and Orai1^{+/-} (31%) animals, and compared with Stim1^{R304W/+} mice (19%), Stim^{R304W/+}Orai1^{+/-} pups were born with a normalized proportion of 27%. Extraction of skeletal muscle RNA and subsequent RT-qPCR evidenced a 50% reduction of Orai1 expression in Orai1^{+/-} and Stim1^{R304W/+}Orai1^{+/-} mice compared with controls (FIG. 1B), while Orai2 and Orai3 expression were comparable across the genotypes (FIG. 1C-D), confirming the deletion of an Orai1 allele and ruling out a compensatory upregulation of its paralogues.

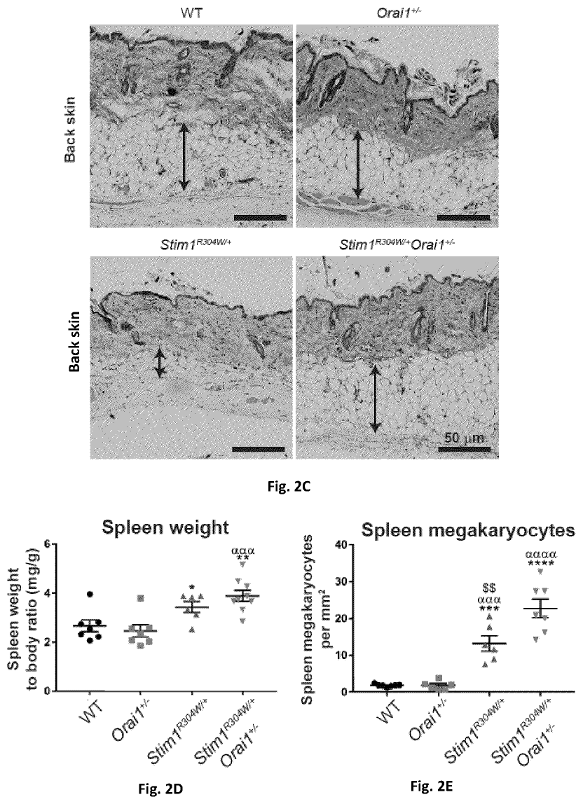

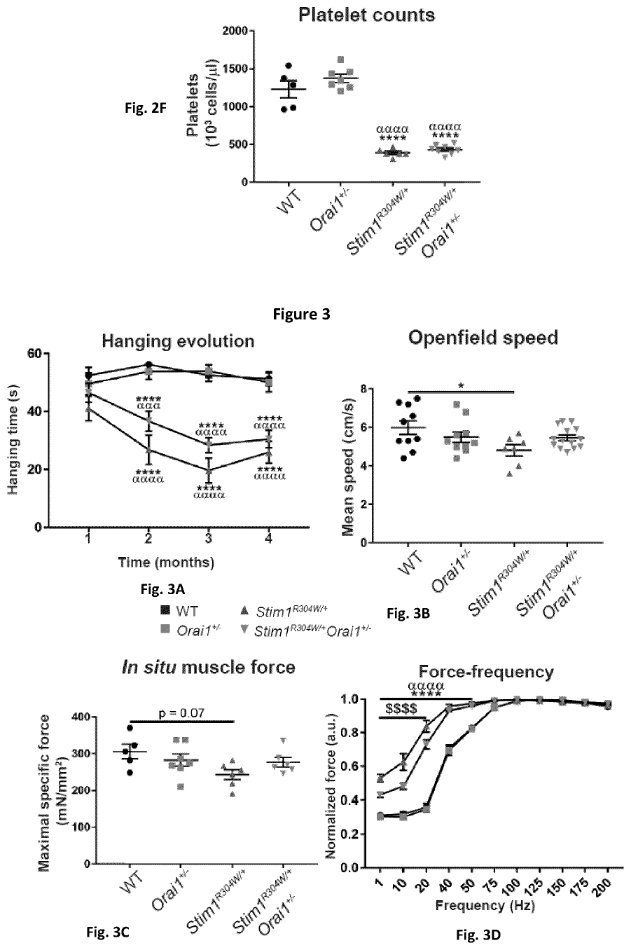

The inventors followed body size and weight development of the offspring over 4 months and in accordance with the previous studies (Silva-Rojas, R. et al., Hum Mol Genet 28, 1579-1593, 2019), the Stim1^{R304W/+} mice showed a distinct growth deficiency in comparison to the control littermates (FIG. 2A and FIG. 1E). At every timepoint of measurement, the Stim1^{R304W/+}Orai^{+/-} mice were significantly bigger and heavier than the Stim1^{R304W/+} mice with a difference of 75 mm and 5 g at 4 months, corresponding to an increase of 23% and 10%, respectively (FIG. 2A). Overall, the present data confirmed the lower birth ratio and weight gain of Stim1^{R304W/+} mice and the absence of an overt deleterious effect of ORAI1 downregulation in Orai1^{+/-} mice. They also suggest that Stim1^{R304W/+}Orai1^{+/-} offspring overcome the risk of prenatal lethality and document an ameliorated postnatal development of the TAM/STRMK animals with reduced Orai1 expression.

### Example 2: Improved Bone Architecture and Muscle Weight in Stim1^{R304W/+}Orai1^{+/-} Mice

The continuous growth of organisms from birth to adulthood is intrinsically linked to the counterbalance of bone-forming osteoblasts and bone-resorbing osteoclasts, and the proliferation and differentiation of both osteoblasts and osteoclasts is SOCE-dependent. Consistently, Stim1^{R304W/+} bones were shown to exhibit structural anomalies of the bones (Silva-Rojas, R. et al., Hum Mol Genet 28, 1579-1593, 2019), presumably accounting for the short stature of TAM/STRMK patients and mice. To determine if the ameliorated growth curves of Stim1^{R304W/+}Orai1^{+/-} mice correlate with a rectified bone architecture, the inventors performed micro-computerized tomography and 3D representations. Bones from Stim1^{R304W/+}Orai1^{+/-} animals showed an improved cortical and trabecular texture and strength compared with Stim1^{R304W/+} mice as illustrated by a significant increased moment of inertia (MOI) of 33% and a reduced trabecular separation of 43% of tibia and femur, respectively (FIG. 2B and Tables 1-2).

TABLE 1
Trabecular bone parameters of femur.

| | BV/TV | Tb. Th | Tb. N | Tb. Sp |
|---|---|---|---|---|
| | (%) | (μm) | (1/mm) | (μm) |
| WT | 7.61 ± | 68.71 ± | 1.08 ± | 377.60 ± |
| | 1.36 | 3.01 | 0.15 | 40.76 |
| Orai1^{+/-} | 4.74 ± | 60.93 ± | 0.73 ± | 390.86 ± |
| | 1.21 | 4.54 | 0.14 | 22.18 |
| Stim1^{R304W/+} | 0.72 ± | 51.60 ± | 0.13 ± | 781.76 ± |
| | 0.25 | 3.46 | 0.04 | 24.58 |
| Stim1^{R304W/+} | 3.60 ± | 62.07 ± | 0.55 ± | 520.74 ± |
| Orai1^{+/-} | 0.79 | 2.96 | 0.10 | 36.42 |
| p value | 0.1883 | 0.4805 | 0.0645 | <0.0001 |
| disease vs | | | | |
| therapy | | | | |

TABLE 2
Cortical bone parameters of midshaft tibia.

| | Ct. Th | T. Ar | B. Ar | BA/TA | M. Ar | MOI |
|---|---|---|---|---|---|---|
| | (mm) | (mm²) | (mm²) | (%) | (mm²) | (mm⁴) |
| WT | 0.25 ± | 0.91 ± | 0.74 ± | 81.09 ± | 0.17 ± | 0.15 ± |
| | 0.01 | 0.03 | 0.03 | 0.47 | 0.00 | 0.01 |
| Orai1^{+/-} | 0.23 ± | 0.84 ± | 0.67 ± | 79.48 ± | 0.17 ± | 0.13 ± |
| | 0.01 | 0.04 | 0.04 | 0.56 | 0.00 | 0.01 |
| Stim1^{R304W/+} | 0.22 ± | 0.73 ± | 0.57 ± | 78.64 ± | 0.15 ± | 0.09 ± |
| | 0.01 | 0.03 | 0.03 | 0.83 | 0.00 | 0.00 |
| Stim1^{R304W/+}Orai1^{+/-} | 0.25 ± | 0.84 ± | 0.68 ± | 80.98 ± | 0.16 ± | 0.12 ± |
| | 0.01 | 0.02 | 0.02 | 0.32 | 0.00 | 0.00 |
| p value | 0.0348 | 0.0640 | 0.0575 | 0.0304 | >0.9999 | 0.1488 |
| disease vs therapy | | | | | | |

Skeletal muscles are attached to bones to effectuate voluntary movements, and bone growth comes along with the buildup of muscle mass. The ratio of small oxidative type I and large glycolytic type II fibers adapts individual muscles to either powerful movements or endurance activities, and the interconversion between type I and type 11 fibers is Ca²⁺-dependent. As a result of abnormal SOCE, muscles from Stim1^{R304W/+} mice showed a fiber type shift associated with hypotrophy of the mixed gastrocnemius, and hypertrophy of the soleus muscle, essentially composed of slow-twitch type I fibers. To determine a potential effect of Orai1 downregulation on muscle mass, the inventors dissected and weighted Stim1^{R304W/+} and Stim1^{R304W/+}Orai1^{+/-} muscles at 4 months of age. While no difference was observed for the gastrocnemius, the soleus was 17% lighter in Stim1^{R304W/+}Orai^{+/-} mice (FIG. 1F-G). Altogether, the analysis of the factors contributing to the ameliorated postnatal development of Stim1^{R304W/+}Orai1^{+/-} mice revealed an improved bone structure and a partial normalization of muscle weight.

### Example 3: Unchanged skin, spleen, and platelet phenotypes in Stim1^{R304W/+} Orai1^{+/-} mice

Skin anomalies including ichthyosis, eczema, or anhidrosis are common features of TAM/STRMK. Histological analyses of patient samples disclosed an obstruction of the eccrine glands, resulting in sweat retention and representing a risk factor for associated skin irritations, and Stim1^{R304W/+} mice displayed an enlarged dermis and a thinning of the subcutaneous fat layer. To evaluate the impact of Orai1 downregulation on the dermal composition, the inventors examined cross sections of Stim1^{R304W/+} and Stim1^{R304W/+}Orai1^{+/-} skin samples. Although single Stim1^{R304W/+}Orai1^{+/-} mice showed a distinct increase of the fat layer area, no overall significant difference was measurable compared with Stim1^{R304W/+} mice (FIG. 2C and FIGS. 1H-1).

Another hallmark of TAM/STRMK is spleen dysfunction in combination with thrombocytopenia and bleeding diathesis. Alike the human phenotype, Stim1^{R304W/+} mice showed morphological spleen anomalies and a reduction of the total platelet number by 70%¹⁸, resulting in reduced thrombus formation upon injury and in increased bleeding times. Stim1^{R304W/+}Orai1^{+/-} animals also manifested splenomegaly and even a more prominent hyperplasia of the megakaryocytes, the precursor cells forming and releasing platelets to the bloodstream (FIG. 2C-D). In compliance with the uncorrected spleen phenotype, platelet counts were similarly low in Stim1^{R304W/+} and Stim1^{R304W/+}Orai^{+/-} animals (FIG. 2E), indicating that the downregulation of Orai1 by 50% has no reversing effect on the spleen and platelet anomalies characterizing TAM/STRMK.

### Example 4: Improved muscle contraction properties in Stim1^{R304W/+}Orai1^{+/-} mice

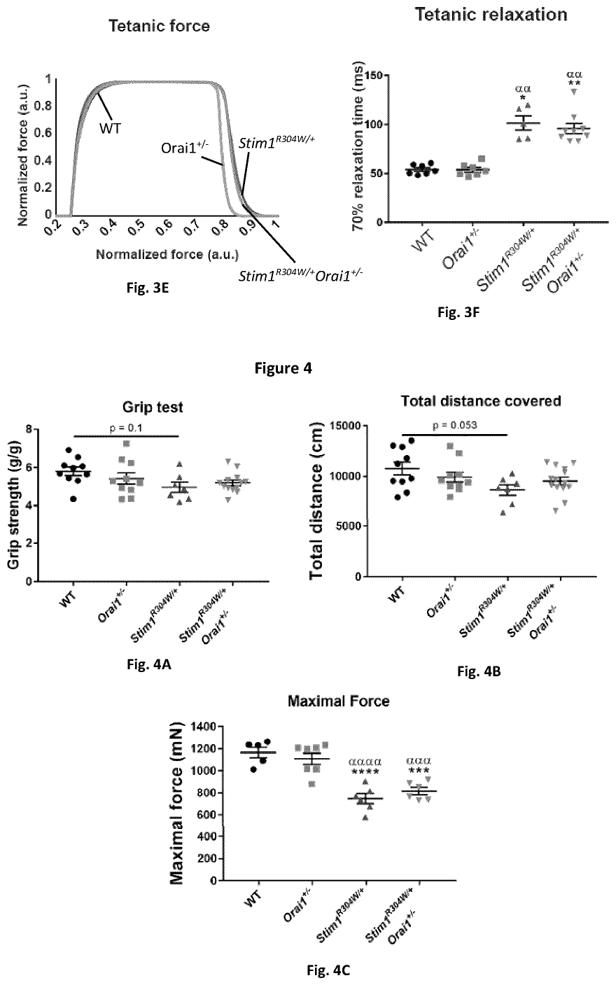

Muscle weakness and exercise intolerance are the principal disabling traits of TAM/STRMK. Affected individuals have difficulties climbing stairs, running, or standing up from a squatting position, and consistently, Stim1^{R304W/+} mice manifested deficiencies in general and specific muscle force. To assess a potential improvement of muscle performance through Orai1 downregulation, Stim1^{R304W/+}Orai^{+/-} and control mice underwent hanging, grip, and open field tests complemented by force transduction experiments. Compared with their Stim1^{R304W/+} littermates, Stim1^{R304W/+}Orai1^{+/-} mice showed a non-significant tendency of increased hanging times throughout the first 4 months (FIG. 3A), increased grip strength at 4 months, and higher mean speed and covered distance in the open field at 3 months (FIG. 3B and FIGS. 4A-B). In situ muscle force measurements on tibialis anterior at 8 months of age confirmed the slight but not significant improvement of maximal and specific muscle force of Stim1^{R304W/+}Orai1^{+/-} compared with Stim1^{R304W/+} mice (FIGS. 3C and 4C).

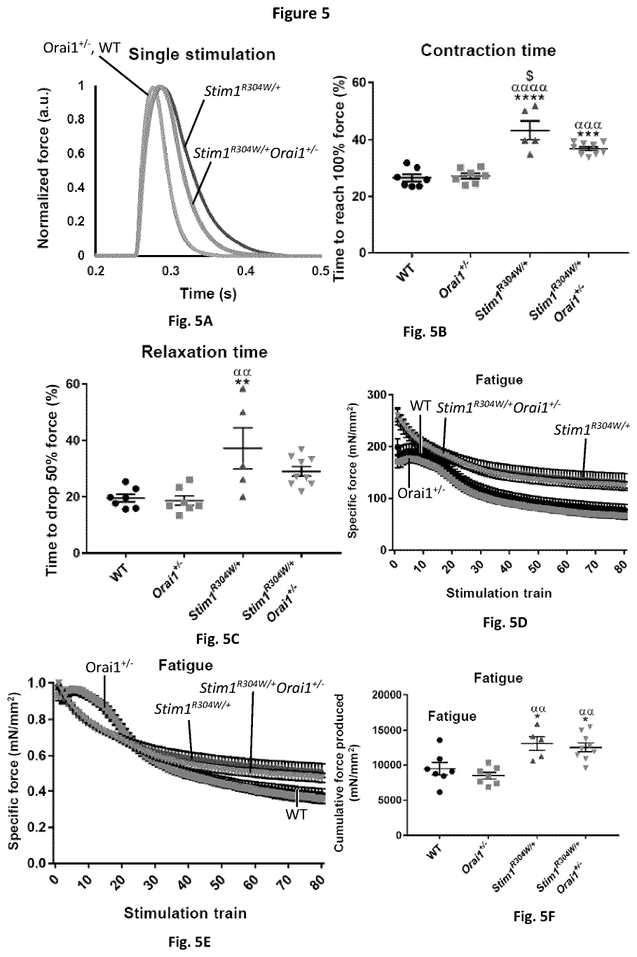

Muscle contraction is a multistep process initiated by an electrical stimulus and mediated by the release of Ca²⁺ from the SR. The Ca² ions trigger the shortening of the contractile units to generate force³³, and Ca²⁺ store refill through the ATP-dependent SERCA pumps enables muscle relaxation and maintains high Ca²⁺ gradients across the SR membrane to allow repetitive tetanic stimulations and counteract the effects of fatigue. As a consequence of SOCE over-activation assumedly entailing an abundance of Ca²⁺ at the contractile units, Stim1^{R304W/+} mice manifested an increased force production at low stimulation frequencies together with a delay in muscle contraction/relaxation and abnormal fatigue profiles. In Stim1^{R304W/+}Orai1^{+/-} mice, the force production between 1 and 20 Hz and the muscle contraction kinetics following a single impulse distinctively and significantly shifted towards normal values (FIG. 3D-F and FIGS. 5A-B), and the inventors also noted a non-significant tendency of ameliorated muscle relaxation (FIGS. 5A and 5C). The fatigue curves following repetitive stimulations however remained identical in Stim1^{R304W/+}Orai1^{+/-} and Stim1^{R304W/+} mice (FIG. 5D-G). In summary, the reduction of Orai1 expression by half has measurable and partially significant effects on muscle force and functionality of Stim1^{R304W/+}Orai1^{+/-} mice.

### Example 5: Normalized Muscle Fiber Size and Improved Autophagic Flux in Stim1^{R304W/+}Orai1^{+/-} Mice

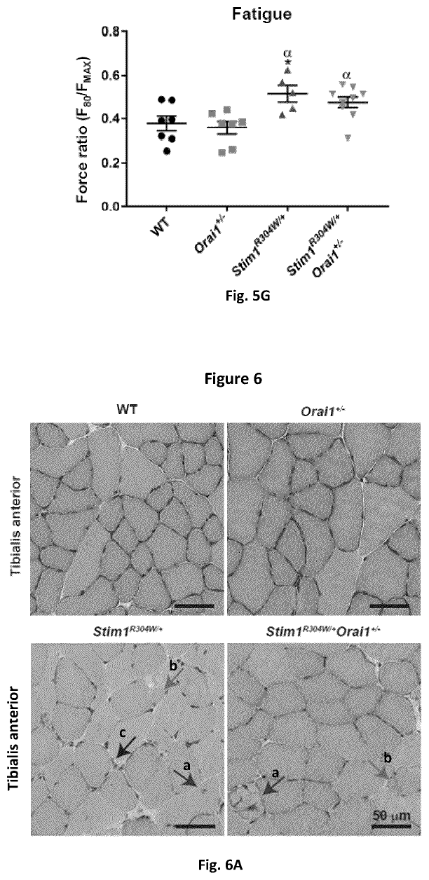

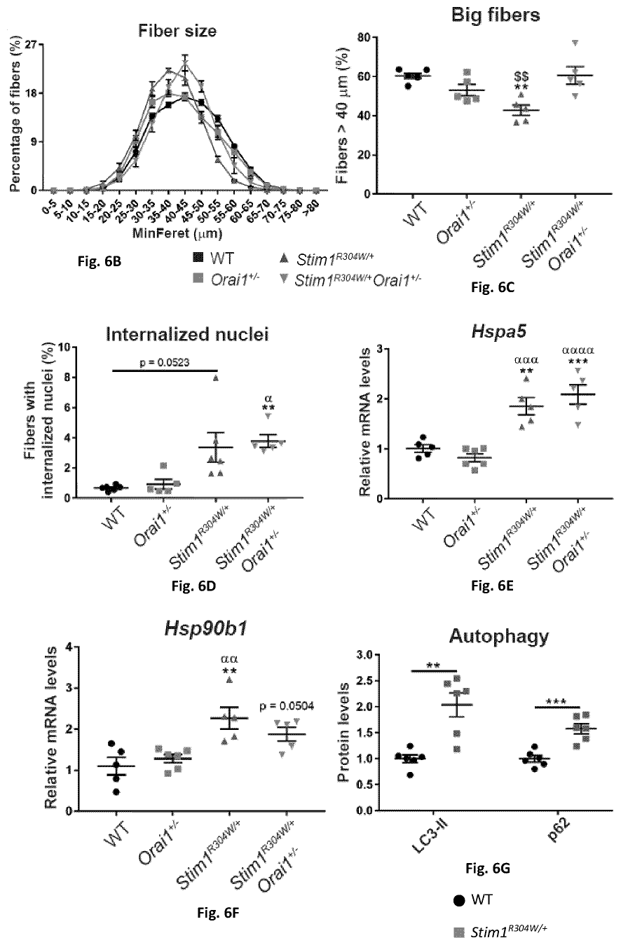

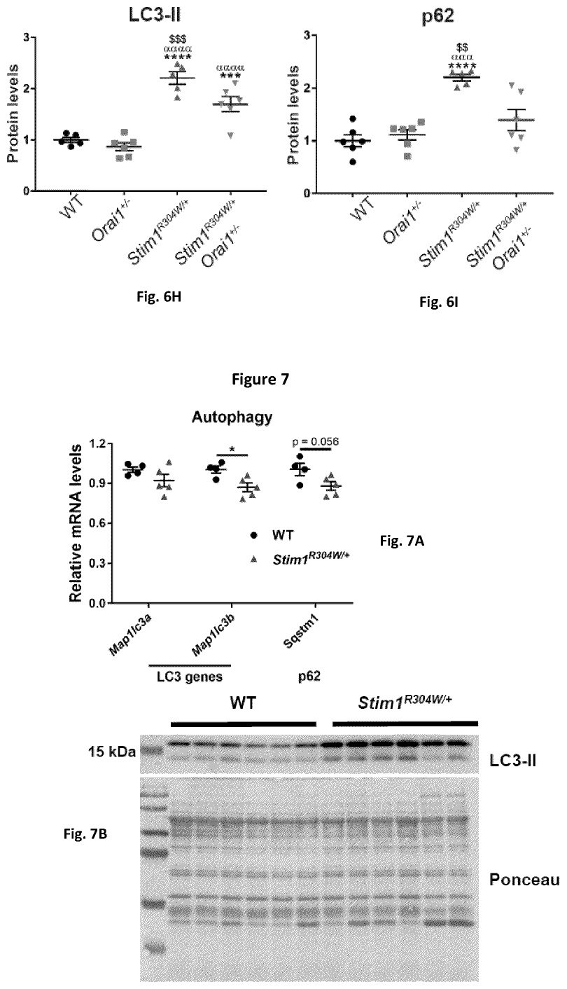

Muscle weakness in TAM/STRMK mice is accompanied by signs of muscle fiber degeneration and regeneration on biopsies such as fiber atrophy, nuclear centralization, and infiltration of immune cells. To determine if the improved muscle performance of Stim1^{R304W/+}Orai1^{+/-} mice bears on an ameliorated muscle structure, the inventors performed histological analyses on transverse tibialis anterior sections. Stim1^{R304W/+}Orai^{+/-} muscle samples displayed an overall enlargement of fiber caliber with 61% of the fibers exceeding a MinFeret diameter of 40 μm compared to 43% in Stim1^{R304W/+} littermates (FIG. 6A-C). The number of fibers with central nuclei was however not reduced in Stim1^{R304W/+}Orai1^{+/-} tibialis anterior, indicating that muscle fiber degeneration was not fully resolved despite the increase of fiber size (FIG. 6D).

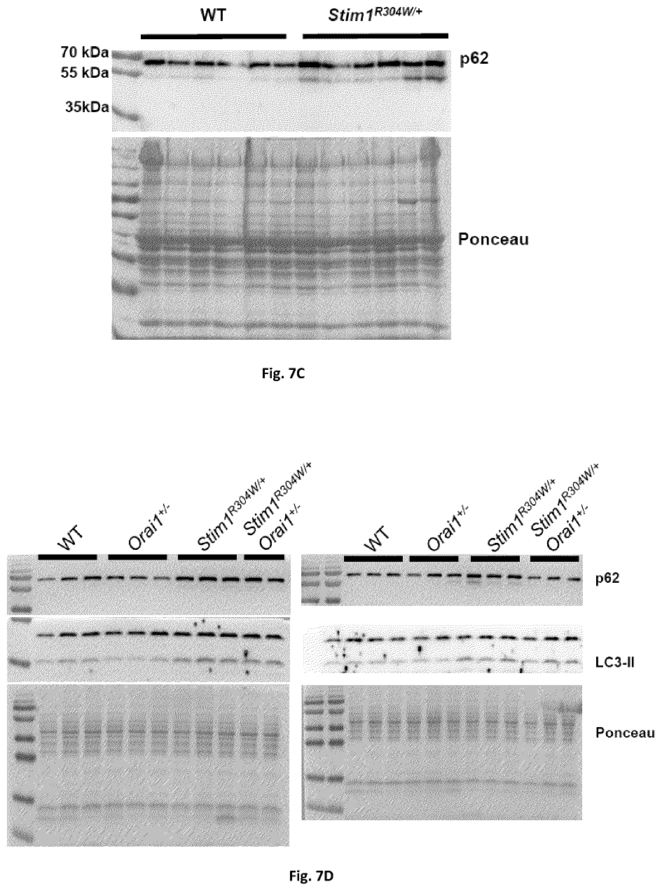

Muscle fiber degeneration in Stim1^{R304W/+} mice results from Ca²⁺-induced reticular stress and the activation of unfolded protein response (UPR) and apoptosis pathways. RT-qPCR on selected UPR markers revealed a comparable upregulation of the chaperones Hsp5 and Hsp90b1 in the tibialis anterior of both Stim1^{R304W/+} and Stim1^{R304W/+}Orai1^{+/-} animals (FIG. 6E-F), suggesting that reticular stress is not resolved in Stim1^{R304W/+}Orai1^{+/-} muscle and accounts for the observed muscle fiber degeneration. To explore the pathomechanisms underlying the increase of myofiber diameter in Stim1^{R304W/+}Orai1^{+/-} mice, the inventors next addressed autophagy, an organelle recycling pathway implicated in the regulation of muscle mass³⁶. The inventors detected a decreased expression of the main autophagy genes Map1lc3a, Map1lc3a, and Sqstm1 in Stim1^{R304W/+} mice compared with the WT (FIG. 7A), while western blots on muscle extracts revealed an increased level of the autophagosome components LC3 1 and p62 (FIG. 6G and FIG. 7B-C), indicating enhanced autophagosome formation or impaired fusion with the lysosome and suggesting a bock of late-stage autophagy. Noteworthy, the LC3 II and p62 levels were significantly reduced in Stim1^{R304W/+}Orai1^{+/-} tibialis anterior compared with Stim1^{R304W/+} mice (FIGS. 6H-1 and 7D), indicating a recovery of the autophagic flux through Orai1 downregulation and providing a potential explanation for the increase in muscle fiber diameter despite continued UPR and myofiber degeneration.

### Example 6: shRNA-Driven Orai1 Silencing Partially Reverses the Muscle Phenotype of Stim1^{R304W/+} Mice

The crossing experiments on the TAM/STRMK mouse model and the survey of birth ratio, growth, and bone, skin, spleen, platelet, and muscle phenotypes of the Stim1^{R304W/+}Orai1^{+/-} offspring and control littermates provided the proof-of-concept that decreased Orai1 expression efficiently anticipates full disease development with a discernible impact on skeletal muscle function and structure. In order to establish an appropriate and applicable procedure to specifically downregulate Orai1 in postnatal tissues, the inventors used RNA interference.

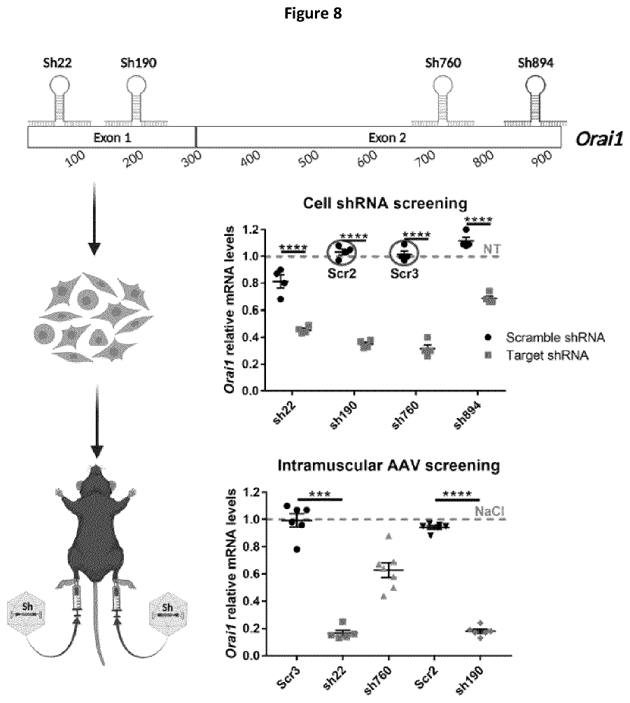

The inventors aligned the mouse Orai1 sequence with its paralogues Orai2 and Orai3, and the inventors designed four shRNAs targeting stretches of 19 to 22 Orai1-specific nucleotides largely conserved in humans (FIG. 8). Transfection of murine C2C12 myoblasts and subsequent RNA extraction and RT-qPCR demonstrated an Orai1 downregulation of least 50% through shRNAs sh22, sh190 and sh760 compared with untransfected controls or cells expressing scramble shRNAs (FIG. 8). To validate Orai1 silencing in vivo, the inventors generated AAV9 particles containing the shRNAs and injected the tibialis anterior of 1-month-old WT mice. Four weeks post-injection, sh22 and sh190 yielded an Orai1 downregulation of more than 80% as compared to NaCl-injected control muscles, while sh760 was less efficient and therefore discarded (FIG. 8).

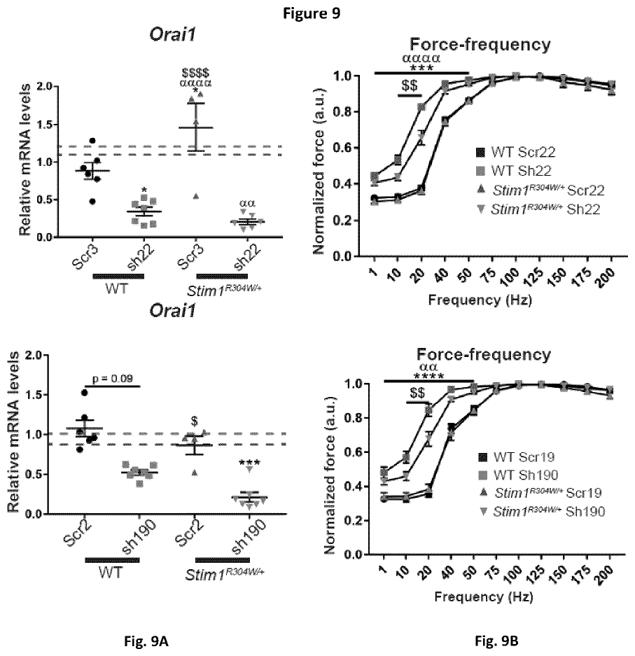

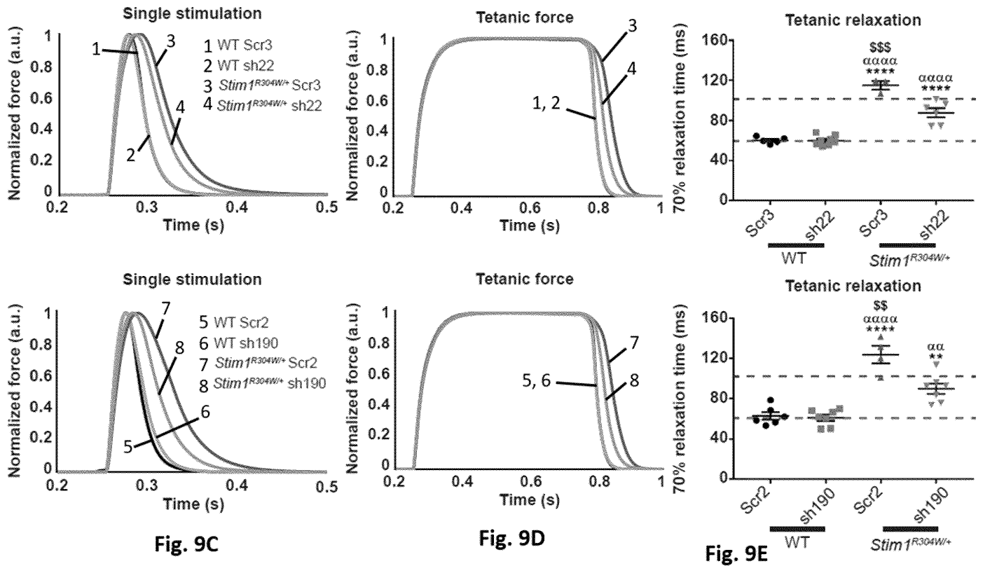

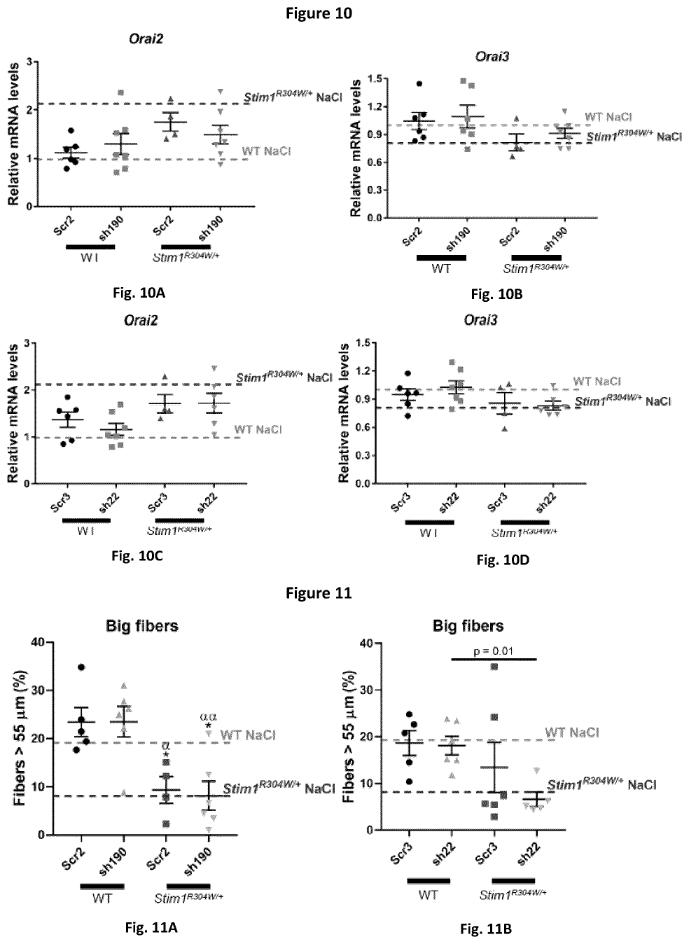

To determine the ability of the selected shRNAs to reverse the muscle defects of TAM/STRMK, the inventors proceeded with the intramuscular AAV injection of either sh22 or sh190 in WT and Stim1^{R304W/+} mice at 2 months of age, and the inventors investigated muscle function, structure, and physiology 2 months post injection. Orai1 downregulation ranged from 50% to 80% (FIG. 9A), whereas the expression levels of Orai2 and Orai3 were comparable in the shRNA-injected, NaCl-injected, and scramble-injected muscles (FIG. 10A-D), demonstrating high specificity of the shRNAs. In situ measurements on anesthetized animals showed a positive effect of both sh22 and sh190 on the force production at low stimulation frequencies of Stim1^{R304W/+} mice compared with the scramble shRNAs, while the muscle contraction properties did not vary between shRNA-injected and NaCl-injected WT mice, excluding a negative impact of the shRNAs on normal muscle function (FIG. 9B). The inventors also observed a significant improvement of the muscle relaxation kinetics with reduced relaxation times in Stim1^{R304W/+} tibialis anterior injected with sh22 and sh190 following single and tetanic stimulations (FIG. 9C-E).

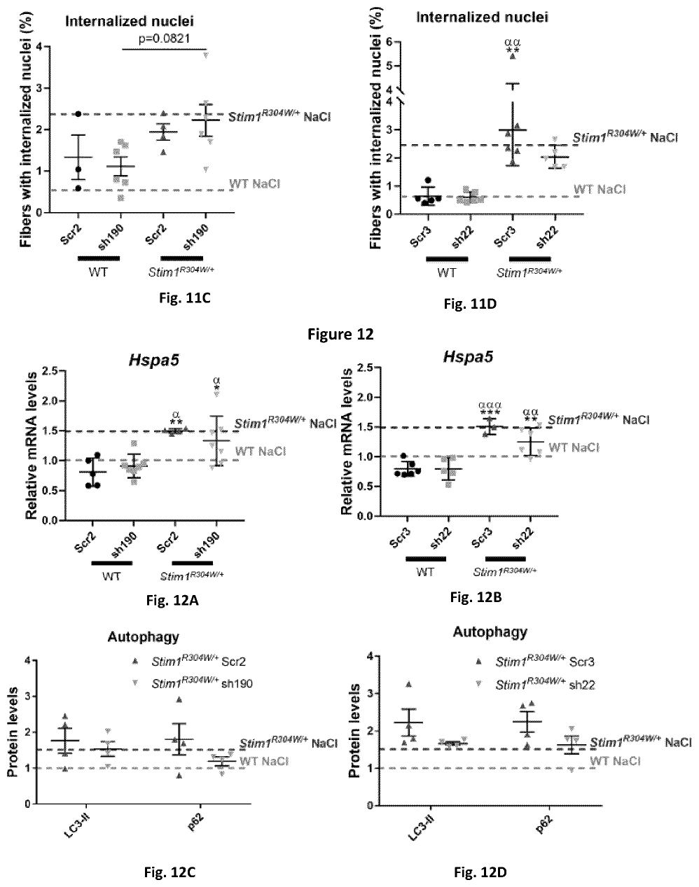

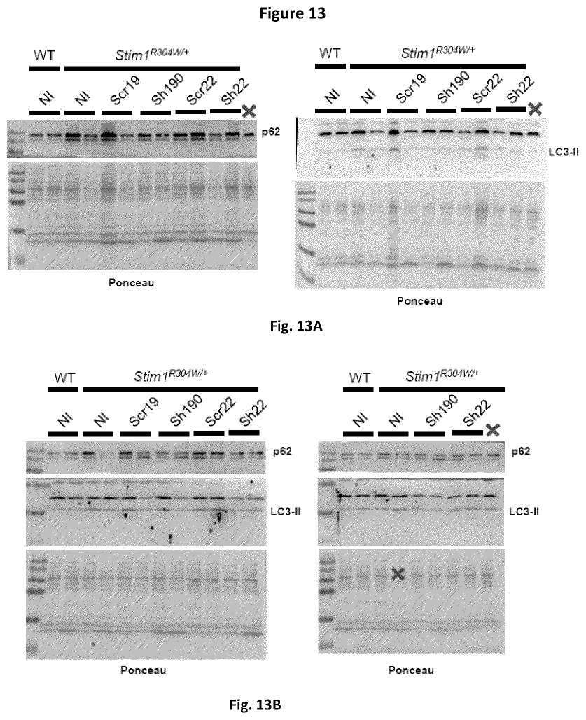

Histological examination of Stim1^{R304W/+} tibialis anterior sections failed to disclose ameliorations of the muscle structure following shRNA delivery. The proportion of fibers with a MinFeret diameter of >55 μM and the number of fibers with centralized nuclei were comparable in shRNA and scramble-injected Stim1^{R304W/+} muscles (FIG. 11A-D). In agreement with the morphological findings, there was no difference in the expression levels of UPR and autophagy markers in Stim1^{R304W/+} tibialis anterior treated with sh22, sh190, or scramble shRNAs (FIGS. 12A-D and 13A). Overall, the shRNA-mediated downregulation of Orai1 did not resolve reticular Ca³⁺ stress and autophagy block, but significantly improved muscle contraction and relaxation properties of the murine TAM/STRMK model.

### Materials and Methods

### Animals

Animal care and experimentation was in accordance with French and European legislation and approved by the institutional ethics committee (project numbers 2019062813376603, 2020052517411298, 2019103108289018 and 2020012813132770). Mice were housed in ventilated cages with 12h day/night cycles and access to food and water ad libitum. Stim1^{R304W/+} and Orai1^{+/-} mice were described previously (Ahuja, Schwartz et al. 2017, Silva-Rojas, Treves et al. 2019); Ahuja, M. et al., Cell Metab 25, 635-646, 2017) and the Orai1^{+/-} mice were a kind gift of Paul F. Worley (Johns Hopkins University, Baltimore, USA). Crossing of both mouse lines resulted in four genotypes: WT, Orai1^{+/-}, Stim1^{R304W/+}, and Stim1^{R304W/+}Orai1^{+/-}. Following primers were used for genotyping: GCAGGTAGGAGAGTGTACAGGATGCCTT (SEQ ID NO: 8) (forward) and CTTTCCATCCCCACTGCCATTTT (SEQ ID NO: 9) (reverse) for Stim1, and ATGCCTACTGCCAAAATTGAC (SEQ ID NO: 10) (forward) and AAATACTGAGCCATCTCTCCTG (SEQ ID NO: 11) (reverse) for Orai1.

### Hanging, Grip, and Open Field Tests

To assess general muscle force, mice were suspended upside down to a cage grid for a maximum of 60 s, and the hanging time was recorded. The four-paw grip strength was measured using a dynamometer (Bioseb, Vitrolles, France) and normalized to body weight. Both hanging and grip tests were performed in triplicate with a 5-10 min rest interval. Hanging time was determined monthly and grip strength once at 4 months of age.

The open field test was performed on 3 months old mice in a homogenously illuminated arena (Bioseb) in a noise-isolated room. Covered distance, speed, and rearing were assessed during 30 min.

### In Situ Muscle Force

To determine maximal and specific muscle force, 4 and 8 months old mice were anesthetized with intraperitoneal injections of domitor/fentanyl mix (2/0.28 mg/Kg), diazepam (8 mg/Kg) and fentanyl (0.28 mg/Kg). The tibialis anterior (TA) was partially excised and the tendon was attached to the isometric transducer of the in situ whole animal system 1305A (Aurora Scientific, Aurora, Canada). Maximal force was determined by sciatic nerve stimulations of 2-200 Hz pulses with an interval of 30 s, and fatigue by 80 stimulations of 40 Hz spaced by 2 s. Specific muscle force was assessed by dividing the maximal force with the TA cross sectional area calculated as wet muscle weight (mg)/optimal muscle length (mm) X mammalian muscle density (1.06 mg/mm³).

### Micro-Computerized Bone Tomography (CT)

Trabecular and cortical bone morphology and structure were assessed on femur and tibia using the Quantum μCT scanner (Perkin Elmer, Waltham, USA). Scans were performed with an isotropic voxel size of 10 μm, 160 μA tube current, and 90 kV tube voltage. Gray scale images were pre-processed using the ImageJ software, and morphological 3D measurements were executed with the CTAn software (Bruker, Billerica, USA). Representative images were generated using the CTvol software (Bruker).

### Bleeding Test and Blood Counts

Mice were anesthetized by inhalation of isoflurane through masks. A distal 10-mm segment of the tail was amputated with a scalpel, and the tail was immediately immersed in 0.9% isotonic PBS solution at 37° C. The bleeding time was defined as the time required until bleeding ceased. The blood-PBS solution underwent OD analysis to determine overall blood loss.

Blood counts were performed on the ADVIA 120 system (Siemens, Munich, Germany) following submandibular puncture under isoflurane anesthesia of 4 and 8 months old mice to determine total platelet, erythrocyte, and leukocyte numbers.

### Muscle, Spleen, and Skin Histology

TA muscles were frozen in liquid nitrogen-cooled isopentane and transverse 8 μm sections were stained with hematoxylin and eosin (H&E), and the Cellpose algorithm (Stringer, C. et al., Nat Methods 18, 100-106, 2021) was used to segment and delineate the individual myofibers. The MinFeret diameter was calculated using ImageJ, and the number of fibers with internal nuclei was determined through the Cell Counter ImageJ plugin. The spleen and a dorsal skin fragment were fixed in 4% paraformaldehyde for 24 h, embedded in paraffin, and 5 μm sections were stained with H&E. The megakaryocyte number was determined on random images covering 12.3 mm² per spleen using the ImageJ Cell Counter plugin, and the thickness and relative proportion of the subcutaneous fat layer was determined on a 5 mm² skin sample area using the NDP Viewer software (Hamamatsu, Hamamatsu, Japan). All muscle, spleen, and skin section were imaged with the Nanozoomer 2HT slide scanner (Hamamatsu).

### Gene Expression and Protein Studies

Total RNA was extracted from TA samples with TRI Reagent (Molecular Research Center, Cincinnati, USA) and reverse transcribed using the SuperScript™ IV Transcriptase (ThermoFisher Scientific, Waltham, USA). For quantitative PCR, the cDNA was amplified using the SYBR Green Master Mix I (Roche Diagnostics, Basel, Switzerland) on a LightCycler 480 Real-Time PCR System (Roche) with forward and reverse primers (Table 3). Primer specificity was determined through melting curve products followed by Sanger sequencing of the amplicons. Rpl27 was used as reference gene (Thomas, K. C. et al., PLoS One 9, e88653, 2014).

TABLE 3
List of primers used for RT-qPCR.

| | | SEQ | | SEQ |
|---|---|---|---|---|
| Gene | Forward primer | ID NO | Reverse Primer | ID NO |
| Rpl27 | AAGCCGTCATCGTGAAGAACA | 12 | CTTGATCTTGGATCGCTTGGC | 21 |
| Orai1 | GCCAAGCTCAAAGCTTCC | 13 | CCTGGTGGGTAGTCATGGTC | 22 |
| Orai2 | GCAGCTACCTGGAACTCGTCACG | 14 | GAGGGTACTGGTACTTGGTCTCCA | 23 |
| Orai3 | GGGCCAGTCAGCACTCTC | 15 | AGTGGTGCAGGCACTAAATG | 24 |
| Hspa5 | CTATTCCTGCGTCGGTGTGT | 16 | ATTCCAAGTGCGTCCGATGA | 25 |
| Hspb90b1 | CCACTCAAATCGAACACGGC | 17 | AGATTCCGCCTCCTTTCTGC | 26 |
| Map1lc3a | CTATGAACAGGAGAAGGATGAAG | 18 | ACTCAGAAGCCGAAGGTT | 27 |
| Map1lc3b | CGTCCTGGACAAGACCAAGT | 19 | ATTGCTGTCCCGAATGTCTC | 28 |
| Sqstm1 | CCTTGCCCTACAGCTGAGTC | 20 | CACACTCTCCCCCACATTCT | 29 |

For protein studies, TA cryosections were lysed in RIPA (radio immunoprecipitation) buffer supplemented with 1 mM PMSF, 1 mM DTT and complete mini EDTA-free protease inhibitor cocktail (Roche). The denatured samples were loaded on 10% or 15% SDS-PAGE gels and transferred onto nitrocellulose membranes using the Transblot® Turbo™ RTA Transfer Kit (Biorad, Hercules, USA). Ponceau S staining (Sigma-Aldrich, St Louis, USA) served as loading control. Following primary and secondary antibodies were used: mouse anti-P62 (1/5000; H00008878-M01, Abnova, Taipeh, Taiwan), rabbit anti-LC3 (1/1000; NB100-2220, Novus Biologicals, Littleton, USA), peroxidase-coupled goat anti-mouse (1/10000; 115-036-068, Jackson ImmunoResearch), and peroxidase-coupled goat anti-rabbit (1/10000; 112-036-045, Jackson ImmunoResearch, Ely, UK). Signal intensity was recorded with the Amersham Imager 600 (Amersham, UK).

shRNA Cloning and AAV Production

shRNA sequences were designed to target Orai1 regions conserved in human and mice and diverging from Orai2 and Orai3. For each Orai1 shRNA, scramble shRNAs were calculated using a specific design software (https://www.invivogen.com/sirnawizard/scrambled.php). The shRNAs (Table 4) were subcloned into pENTR1A and cloned into the pAAV plasmid under the control of the U6 promoter and flanked by serotype 2 inverted terminal repeats using the Gateway system (ThermoFisher Scientific). sh190 targets the same 19 nucleotides as the SYL116011 siRNA, developed by Sylentis to treat ocular allergies and conjunctivitis (Gonzalez, V. et al., Invest Ophth Vis Sci 59, 2018; Jimenez, A. I. et al., Invest Ophth Vis Sci 56, 2015).

AAV particles were produced by triple transfection of the HEK293T cell line with pAAV, the helper plasmid, and pXR1 containing rep and cap genes of AAV serotype 9. Cell lysates were treated with 50 U/mL Benzonase (Sigma-Aldrich) for 30 min at 37° C. and clarified by centrifugation. Viral particles were purified by iodixanol gradient ultracentrifugation using Amicon Ultra-15 Centrifucal Filters (Merck, Darmstadt, Germany) and followed by dialysis. Particle quantity was determined by real-time PCR using TACGGTAAACTGCCCACTTG (SEQ ID NO: 30) (forward) and AGGAAAGTCCCATAAGGTCA (SEQ ID NO: 31) (reverse) primers. Titers are expressed as viral genomes per mL (vg/mL).

TABLE 4
Oligos used for shRNA cloning.

| Oligo | |
|---|---|
| name | Sequence |
| Scr1_Fw | AGCTTTGTTTACGACGTCACGGCAGCGATCACTTC |
| | AAGAGAGTGATCGCTGCCGTGACGTCGTCTTTTTA |
| Scr1_Rv | GATCTAAAAAGACGACGTCACGGCAGCGATCACTC |
| | TCTTGAAGTGATCGCTGCCGTGACGTCGTAAACAA |
| Sh22_Fw | AGCTTTGTTTGCCCGAGTCACAGCAATCCGGATTC |
| | AAGAGATCCGGATTGCTGTGACTCGGGCCTTTTTA |
| Sh22_Rv | GATCTAAAAAGGCCCGAGTCAGAGCAATCCGGATC |
| | TCTTGAATCCGGATTGCTGTGACTCGGGCAAACAA |
| Scr19_Fw | AGCTTTGTTTTAGGCGCGTCACGAAGAACTTCAAG |
| | AGAGTTCTTCGTGACGCGCCTACTTTTTA |
| Scr19_Fw | GATCTAAAAAGTAGGCGCGTCACGAAGAACTCTCT |
| | TGAAGTTCTTCGTGACGCGCCTAAAACAA |
| Sh190_Fw | AGCTTTGTTTGGATGAGCCTCAACGAGCATTCAAG |
| | AGATGCTCGTTGAGGCTCATCCCTTTTTA |
| Sh190_Rv | GATCTAAAAAGGGATGAGCCTCAACGAGCATCTCT |
| | TGAATGCTCGTTGAGGCTCATCCAAACAA |
| Scr22_Fw | AGCTTTGTTTTCTTATGCGGTATTCTCTCTTCTTC |
| | AAGAGAGAAGAGAGAATACCGCATAAGACTTTTTA |
| Scr22_Rv | GATCTAAAAAGTCTTATGGGGTATTCTCTCTTCTC |
| | TCTTGAAGAAGAGAGAATACCGCATAAGAAAACAA |
| Sh760_Fw | AGCTTTGTTTATCGTCTTTGCTGTTCACTTCTTTC |
| | AAGAGAAGAAGTGAACAGCAAAGACGATCTTTTTA |
| Sh760_Rv | GATCTAAAAAGATCGTCTTTGCTGTTCACTTCTTC |
| | TCTTGAAAGAAGTGAACAGCAAAGACGATAAACAA |
| Scr4_Fw | AGCTTTGTTTGACCACACAGTCGCGCTATACCTTC |
| | AAGAGAGGTATAGCGCGACTGTGTGGTCCTTTTTA |
| Scr4_Rv | GATCTAAAAAGGACCACACAGTCGCGCTATACCTC |
| | TCTTGAAGGTATAGCGCGACTGTGTGGTCAAACAA |
| Sh894_Fw | AGCTTTGTTTACCGGGCACCCACTATGCCTAATTC |
| | AAGAGATTAGGCATAGTGGGTGCCCGGTCTTTTTA |
| Sh894_Rv | GATCTAAAAAGACCGGGCACCCACTATGCCTAATC |
| | TCTTGAATTAGGCATAGTGGGTGCCCGGTAAACAA |

Scr1_Fw: SEQ ID NO: 32; Scr1_Rv: SEQ ID NO: 33; sh22_Fw: SEQ ID NO: 34; sh22_Rv: SEQ ID NO: 35; Scr19_Fw: SEQ ID NO: 36; Scr19_Rv: SEQ ID NO: 37; sh190_Fw: SEQ ID NO: 38; sh190_Rv: SEQ ID NO: 39; scr22_Fw: SEQ ID NO: 40; scr22_Rv: SEQ ID NO: 41; sh760_Fw: SEQ ID NO: 42; sh760_Rv: SEQ ID NO: 43; scr4_Fw: SEQ ID NO: 44; scr4_Rv: SEQ ID NO: 45; sh894_Fw: SEQ ID NO: 46; sh894_Rv: SEQ ID NO: 47.

shRNA Screening and Intramuscular AAV Injection

For the cellular shRNA screening, pENTR1A plasmids were transfected into C2C12 myoblasts using Lipofectamine 3000 (Invitrogen, Waltham, USA). Cells were harvested after 48 h to extract RNA and quantify Orai1 expression. For in vivo validation, 1 month old WT mice were anesthetized by intraperitoneal injection of ketamine 100 g/g and xylazine 5 g/g of body weight. TAs were injected with 1.2×10¹⁰ viral genomes/TA or 20 μL of NaCl 0.9% as control. At 2 months of age, the animals were euthanized, and Orai1 silencing in TA samples was assessed by RT-qPCR.

To evaluate the therapeutic potential of the shRNAs, 2 months old WT and Stim1^{R304W/+} mice were anesthetized and randomly injected with 1.5×10¹⁰ viral genomes/TA or 25 μL of NaCl 0.9% as control. At 4 months of age, the mice underwent in situ muscle force measurements, and the TAs were dissected for subsequent morphological and gene expression analyses.

### Study Randomization and Statistical Analysis

All experiments were performed and analyzed in a blinded manner and the investigators were unaware of the genotype of the mice. The normal distribution of the data was assessed using the Shapiro-Wilk test and presented as mean±standard error of the mean (SEM). For normally distributed data, the significance of changes was examined by two-tailed Student's t-test with or without Welch's correction for comparison of 2 groups or by one-way ANOVA followed by Tukey's post hoc test for comparison of more than 2 groups. In case of not-normally distributed data, Mann-Whitney test was used to compare 2 groups and Kruskal-Wallis followed by Dunn's multiple comparison test to compare more than 2 groups. The statistical significance of birth ratio was assessed by chi-square test.

1-15. (canceled)

16. A method for treating a disease selected from the group consisting of tubular aggregate myopathy (TAM) and Stormorken syndrome (STRMK) in a subject, comprising administering a therapeutically amount of a shRNA, a nucleic acid encoding said shRNA or a pharmaceutical composition comprising a therapeutically amount of a shRNA or a nucleic acid encoding said shRNA to the subject, said shRNA inhibiting the expression of Calcium release-activated calcium channel protein 1 (ORAI1), thereby improving muscle function.

17. The method according to claim 16, wherein the shRNA specifically targets SEQ ID NO: 5.

18. The method according to claim 17, wherein the nucleic acid encoding the shRNA comprises SEQ ID NO: 6 and/or the shRNA comprises SEQ ID NO: 7.

19. The method according to claim 16, wherein the shRNA specifically targets a sequence selected from SEQ ID NO: 1 and SEQ ID NO: 2.

20. The method according to claim 19, wherein the nucleic acid encoding the shRNA comprises SEQ ID NO: 3 and/or the shRNA comprises SEQ ID NO: 4.

21. The method according to claim 16, wherein the shRNA specifically targets a sequence selected in the group consisting of SEQ ID NOs: 48-156.

22. The method according to claim 16, wherein said nucleic acid encoding said shRNA a naked DNA or is contained in a vector encoding the shRNA.

23. The method according to claim 16, wherein the nucleic acid encoding said shRNA is controlled by an ubiquitous promoter or a promoter specific for muscle.

24. The method according to claim 22, wherein the vector is a viral vector.

25. The method according to claim 24, wherein the viral vector is an adeno-associated vector (AAV), adenoviral vector, baculoviral vector, herpes viral vector, retroviral vector or a lentiviral vector.

26. The method according to claim 25, wherein the vector is an AAV vector.

27. The method according to claim 26, wherein the vector is a viral vector, a plasmid or human artificial chromosomes (HAC).

28. A pharmaceutical composition comprising a shRNA or a nucleic acid encoding said shRNA, wherein the shRNA inhibits the ORAI1 expression and specifically targets a sequence selected from SEQ ID NO: 1 or SEQ ID NO: 2.

29. The pharmaceutical composition according to claim 28, wherein the nucleic acid encoding the shRNA comprises SEQ ID NO: 3 and/or the shRNA has the sequence of SEQ ID NO: 4.

30. The pharmaceutical composition according to claim 28, wherein said nucleic acid encoding said shRNA a naked DNA or is contained in a vector encoding the shRNA.

31. The pharmaceutical composition according to claim 28, wherein the nucleic acid encoding said shRNA is controlled by an ubiquitous promoter or a promoter specific for muscle.

32. The pharmaceutical composition according to claim 30, wherein the vector is a viral vector.

33. The pharmaceutical composition according to claim 32, wherein the viral vector is an adeno-associated vector (AAV), adenoviral vector, baculoviral vector, herpes viral vector, retroviral vector or a lentiviral vector.

34. The pharmaceutical composition according to claim 33, wherein the vector is an AAV vector.

35. The pharmaceutical composition according to claim 30, wherein the vector is a viral vector, a plasmid or human artificial chromosomes (HAC).
